SEQUENCE LISTING (22 sequences)

SEQ ID NO: 1 - DNA, 21 bp, synthetic construct
  source 1..21: mol_type = other DNA
  modified_base 1: mod_base = OTHER; note = 2'-O-methyl modified nucleotide
  modified_base 2: mod_base = OTHER; note = 2'-O-methyl modified nucleotide
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-O-methyl modified nucleotide
  modified_base 7: mod_base = OTHER; note = 2'-fluoro modified nucleotide
  modified_base 8: mod_base = OTHER; note = 2'-O-methyl modified nucleotide
  modified_base 9: mod_base = OTHER; note = 2'-fluoro modified nucleotide
  modified_base 12: mod_base = OTHER; note = 2', 2'-difluoro 2'-deoxycytidine
  modified_base 14: mod_base = OTHER; note = 2'-O-methyl modified nucleotide
  modified_base 15: mod_base = OTHER; note = 2'-O-methyl modified nucleotide
  modified_base 18: mod_base = OTHER; note = 2', 2'-difluoro 2'-deoxycytidine
  modified_base 20: mod_base = OTHER; note = 2'-O-methyl modified nucleotide
  modified_base 21: mod_base = OTHER; note = 2'-O-methyl modified nucleotide
  misc_feature 1..11: note = RNA
  misc_feature 12: note = DNA
  misc_feature 13..17: note = RNA
  misc_feature 18: note = DNA
  misc_feature 19..21: note = RNA
aagaaagagatctgtatcaat

SEQ ID NO: 2 - DNA, 25 bp, synthetic construct
  source 1..25: mol_type = other DNA
  modified_base 1: mod_base = OTHER; note = 2'-O-methyl modified nucleotide
  modified_base 2: mod_base = OTHER; note = 2'-fluoro modified nucleotide
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-fluoro modified nucleotide
  modified_base 7: mod_base = OTHER; note = 2'-O-methyl modified nucleotide
  modified_base 8: mod_base = OTHER; note = 2'-O-methyl modified nucleotide
  modified_base 9: mod_base = OTHER; note = 2'-O-methyl modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-O-methyl modified nucleotide
  modified_base 16: mod_base = OTHER; note = 2'-fluoro modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide
  modified_base 22: mod_base = OTHER; note = 2', 2'-difluoro 2'-deoxycytidine
  modified_base 23: mod_base = OTHER; note = 2', 2'-difluoro 2'-deoxycytidine
  misc_feature 1..21: note = RNA
  misc_feature 22..25: note = DNA
attgatacagatctctttcttcctt

SEQ ID NO: 3 - DNA, 23 bp, synthetic construct
  source 1..23: mol_type = other DNA
  modified_base 1: mod_base = OTHER; note = 2'-O-methyl modified nucleotide
  modified_base 2: mod_base = OTHER; note = 2'-O-methyl modified nucleotide
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-O-methyl modified nucleotide
  modified_base 7: mod_base = OTHER; note = 2'-fluoro modified nucleotide
  modified_base 8: mod_base = OTHER; note = 2'-O-methyl modified nucleotide
  modified_base 9: mod_base = OTHER; note = 2'-fluoro modified nucleotide
  modified_base 10: mod_base = OTHER; note = 2'-fluoro modified nucleotide
  modified_base 11: mod_base = OTHER; note = 2'-fluoro modified nucleotide
  modified_base 12: mod_base = OTHER; note = 2', 2'-difluoro 2'-deoxycytidine
  modified_base 13: mod_base = OTHER; note = 2'-O-methyl modified nucleotide
  modified_base 14: mod_base = OTHER; note = 2'-O-methyl modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-O-methyl modified nucleotide
  modified_base 18: mod_base = OTHER; note = 2', 2'-difluoro 2'-deoxycytidine
  modified_base 19: mod_base = OTHER; note = 2'-O-methyl modified nucleotide
  modified_base 20: mod_base = OTHER; note = 2'-O-methyl modified nucleotide
  modified_base 21: mod_base = OTHER; note = 2'-O-methyl modified nucleotide
  misc_feature 1..11: note = RNA
  misc_feature 12: note = DNA
  misc_feature 13..17: note = RNA
  misc_feature 18: note = DNA
  misc_feature 19..21: note = RNA
  misc_feature 22..23: note = DNA
aagaaagagatctgtatcaattt

SEQ ID NO: 4 - DNA, 23 bp, synthetic construct
  source 1..23: mol_type = other DNA
  modified_base 1: mod_base = OTHER; note = 2'-O-methyl modified nucleotide
  modified_base 2: mod_base = OTHER; note = 2'-fluoro modified nucleotide
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-fluoro modified nucleotide
  modified_base 7: mod_base = OTHER; note = 2'-O-methyl modified nucleotide
  modified_base 8: mod_base = OTHER; note = 2'-O-methyl modified nucleotide
  modified_base 9: mod_base = OTHER; note = 2'-O-methyl modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-O-methyl modified nucleotide
  modified_base 16: mod_base = OTHER; note = 2'-fluoro modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide
  modified_base 20: mod_base = OTHER; note = 2'-O-methyl modified nucleotide
  modified_base 21: mod_base = OTHER; note = 2'-O-methyl modified nucleotide
  misc_feature 1..21: note = RNA
  misc_feature 22..23: note = DNA
attgatacagatctctttctttt

SEQ ID NO: 5 - DNA, 21 bp, synthetic construct
  source 1..21: mol_type = other DNA
  modified_base 1: mod_base = OTHER; note = 2'-O-methyl modified nucleotide
  modified_base 2: mod_base = OTHER; note = 2'-O-methyl modified nucleotide
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-O-methyl modified nucleotide
  modified_base 7: mod_base = OTHER; note = 2'-fluoro modified nucleotide
  modified_base 8: mod_base = OTHER; note = 2'-O-methyl modified nucleotide
  modified_base 9: mod_base = OTHER; note = 2'-fluoro modified nucleotide
  modified_base 10: mod_base = OTHER; note = 2'-fluoro modified nucleotide
  modified_base 11: mod_base = OTHER; note = 2'-fluoro modified nucleotide
  modified_base 12: mod_base = OTHER; note = 2', 2'-difluoro 2'-deoxycytidine
  modified_base 13: mod_base = OTHER; note = 2'-O-methyl modified nucleotide
  modified_base 14: mod_base = OTHER; note = 2'-O-methyl modified nucleotide
  modified_base 15: mod_base = OTHER; note = 2'-O-methyl modified nucleotide
  modified_base 18: mod_base = OTHER; note = 2', 2'-difluoro 2'-deoxycytidine
  modified_base 20: mod_base = OTHER; note = 2'-O-methyl modified nucleotide
  modified_base 21: mod_base = OTHER; note = 2'-O-methyl modified nucleotide
  misc_feature 1..11: note = RNA
  misc_feature 12: note = DNA
  misc_feature 13..17: note = RNA
  misc_feature 18: note = DNA
  misc_feature 19..21: note = RNA
aagaaagagatctgtatcaat

SEQ ID NO: 6 - DNA, 25 bp, synthetic construct
  source 1..25: mol_type = other DNA
  modified_base 1: mod_base = OTHER; note = 2'-O-methyl modified nucleotide
  modified_base 2: mod_base = OTHER; note = 2'-fluoro modified nucleotide
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-fluoro modified nucleotide
  modified_base 7: mod_base = OTHER; note = 2'-O-methyl modified nucleotide
  modified_base 8: mod_base = OTHER; note = 2'-O-methyl modified nucleotide
  modified_base 9: mod_base = OTHER; note = 2'-O-methyl modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-O-methyl modified nucleotide
  modified_base 16: mod_base = OTHER; note = 2'-fluoro modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide
  modified_base 22: mod_base = OTHER; note = 2', 2'-difluoro 2'-deoxycytidine
  modified_base 23: mod_base = OTHER; note = 2', 2'-difluoro 2'-deoxycytidine
  misc_feature 1..21: note = RNA
  misc_feature 22..25: note = DNA
attgatacagatctctttcttcctt

SEQ ID NO: 7 - DNA, 21 bp, synthetic construct
  source 1..21: mol_type = other DNA
  modified_base 1: mod_base = OTHER; note = 2'-O-methyl modified nucleotide
  modified_base 2: mod_base = OTHER; note = 2'-O-methyl modified nucleotide
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-O-methyl modified nucleotide
  modified_base 7: mod_base = OTHER; note = 2'-fluoro modified nucleotide
  modified_base 8: mod_base = OTHER; note = 2'-O-methyl modified nucleotide
  modified_base 9: mod_base = OTHER; note = 2'-fluoro modified nucleotide
  modified_base 10: mod_base = OTHER; note = 2'-fluoro modified nucleotide
  modified_base 11: mod_base = OTHER; note = 2'-fluoro modified nucleotide
  modified_base 12: mod_base = OTHER; note = 2', 2'-difluoro 2'-deoxycytidine
  modified_base 13: mod_base = OTHER; note = 2'-O-methyl modified nucleotide
  modified_base 14: mod_base = OTHER; note = 2'-O-methyl modified nucleotide
  modified_base 15: mod_base = OTHER; note = 2'-O-methyl modified nucleotide
  modified_base 18: mod_base = OTHER; note = 2', 2'-difluoro 2'-deoxycytidine
  modified_base 20: mod_base = OTHER; note = 2'-O-methyl modified nucleotide
  modified_base 21: mod_base = OTHER; note = 2'-O-methyl modified nucleotide
  misc_feature 1..11: note = RNA
  misc_feature 12: note = DNA
  misc_feature 13..17: note = RNA
  misc_feature 18: note = DNA
  misc_feature 19..21: note = RNA
aagaaagagatctgtatcaat

SEQ ID NO: 8 - DNA, 25 bp, synthetic construct
  source 1..25: mol_type = other DNA
  modified_base 1: mod_base = OTHER; note = 2'-O-methyl modified nucleotide
  modified_base 2: mod_base = OTHER; note = 2'-fluoro modified nucleotide
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-fluoro modified nucleotide
  modified_base 7: mod_base = OTHER; note = 2'-O-methyl modified nucleotide
  modified_base 8: mod_base = OTHER; note = 2'-O-methyl modified nucleotide
  modified_base 9: mod_base = OTHER; note = 2'-O-methyl modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-O-methyl modified nucleotide
  modified_base 16: mod_base = OTHER; note = 2'-fluoro modified nucleotide
  modified_base 17: mod_base = OTHER; note = 2'-fluoro modified nucleotide
  modified_base 18: mod_base = OTHER; note = 2'-fluoro modified nucleotide
  modified_base 19: mod_base = OTHER; note = 2'-fluoro modified nucleotide
  modified_base 20: mod_base = OTHER; note = 2'-fluoro modified nucleotide
  modified_base 21: mod_base = OTHER; note = 2'-fluoro modified nucleotide
  modified_base 22: mod_base = OTHER; note = 2', 2'-difluoro 2'-deoxycytidine
  modified_base 23: mod_base = OTHER; note = 2', 2'-difluoro 2'-deoxycytidine
  misc_feature 1..21: note = RNA
  misc_feature 22..25: note = DNA
attgatacagatctctttcttcctt

SEQ ID NO: 9 - DNA, 21 bp, synthetic construct
  source 1..21: mol_type = other DNA
  modified_base 1: mod_base = OTHER; note = 2'-O-methyl modified nucleotide
  modified_base 2: mod_base = OTHER; note = 2'-O-methyl modified nucleotide
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-O-methyl modified nucleotide
  modified_base 7: mod_base = OTHER; note = 2'-fluoro modified nucleotide
  modified_base 8: mod_base = OTHER; note = 2'-O-methyl modified nucleotide
  modified_base 9: mod_base = OTHER; note = 2'-fluoro modified nucleotide
  modified_base 10: mod_base = OTHER; note = 2'-fluoro modified nucleotide
  modified_base 11: mod_base = OTHER; note = 2'-fluoro modified nucleotide
  modified_base 12: mod_base = OTHER; note = 2', 2'-difluoro 2'-deoxycytidine
  modified_base 13: mod_base = OTHER; note = 2'-fluoro modified nucleotide
  modified_base 14: mod_base = OTHER; note = 2'-O-methyl modified nucleotide
  modified_base 15: mod_base = OTHER; note = 2'-fluoro modified nucleotide
  modified_base 16: mod_base = OTHER; note = 2'-O-methyl modified nucleotide
  modified_base 17: mod_base = OTHER; note = 2'-fluoro modified nucleotide
  modified_base 18: mod_base = OTHER; note = 2', 2'-difluoro 2'-deoxycytidine
  modified_base 19: mod_base = OTHER; note = 2'-O-methyl modified nucleotide
  modified_base 20: mod_base = OTHER; note = 2'-O-methyl modified nucleotide
  modified_base 21: mod_base = OTHER; note = 2'-fluoro modified nucleotide
  misc_feature 1..11: note = RNA
  misc_feature 12: note = DNA
  misc_feature 13..17: note = RNA
  misc_feature 18: note = DNA
  misc_feature 19..21: note = RNA
aagaaagagatctgtatcaat

SEQ ID NO: 10 - DNA, 25 bp, synthetic construct
  source 1..25: mol_type = other DNA
  modified_base 1: mod_base = OTHER; note = 2'-O-methyl modified nucleotide
  modified_base 2: mod_base = OTHER; note = 2'-fluoro modified nucleotide
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-fluoro modified nucleotide
  modified_base 7: mod_base = OTHER; note = 2'-O-methyl modified nucleotide
  modified_base 8: mod_base = OTHER; note = 2'-O-methyl modified nucleotide
  modified_base 9: mod_base = OTHER; note = 2'-O-methyl modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-fluoro modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-O-methyl modified nucleotide
  modified_base 16: mod_base = OTHER; note = 2'-fluoro modified nucleotide
  modified_base 17: mod_base = OTHER; note = 2'-O-methyl modified nucleotide
  modified_base 18: mod_base = OTHER; note = 2'-fluoro modified nucleotide
  modified_base 19: mod_base = OTHER; note = 2'-O-methyl modified nucleotide
  modified_base 20: mod_base = OTHER; note = 2'-fluoro modified nucleotide
  modified_base 22: mod_base = OTHER; note = 2', 2'-difluoro 2'-deoxycytidine
  modified_base 23: mod_base = OTHER; note = 2', 2'-difluoro 2'-deoxycytidine
  misc_feature 1..21: note = RNA
  misc_feature 22..25: note = DNA
attgatacagatctctttcttcctt

SEQ ID NO: 11 - DNA, 21 bp, synthetic construct
  source 1..21: mol_type = other DNA
  modified_base 1: mod_base = OTHER; note = 2'-O-methyl modified nucleotide
  modified_base 2: mod_base = OTHER; note = 2'-O-methyl modified nucleotide
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-O-methyl modified nucleotide
  modified_base 7: mod_base = OTHER; note = 2'-fluoro modified nucleotide
  modified_base 8: mod_base = OTHER; note = 2'-O-methyl modified nucleotide
  modified_base 9: mod_base = OTHER; note = 2'-fluoro modified nucleotide
  modified_base 10: mod_base = OTHER; note = 2'-fluoro modified nucleotide
  modified_base 11: mod_base = OTHER; note = 2'-fluoro modified nucleotide
  modified_base 12: mod_base = OTHER; note = 2', 2'-difluoro 2'-deoxycytidine
  modified_base 13: mod_base = OTHER; note = 2'-fluoro modified nucleotide
  modified_base 14: mod_base = OTHER; note = 2'-O-methyl modified nucleotide
  modified_base 15: mod_base = OTHER; note = 2'-fluoro modified nucleotide
  modified_base 16: mod_base = OTHER; note = 2'-O-methyl modified nucleotide
  modified_base 17: mod_base = OTHER; note = 2'-fluoro modified nucleotide
  modified_base 18: mod_base = OTHER; note = 2', 2'-difluoro 2'-deoxycytidine
  modified_base 19: mod_base = OTHER; note = 2'-O-methyl modified nucleotide
  modified_base 20: mod_base = OTHER; note = 2'-O-methyl modified nucleotide
  modified_base 21: mod_base = OTHER; note = 2'-fluoro modified nucleotide
  misc_feature 1..11: note = RNA
  misc_feature 12: note = DNA
  misc_feature 13..17: note = RNA
  misc_feature 18: note = DNA
  misc_feature 19..21: note = RNA
cagggtatcgacgattacaaa

SEQ ID NO: 12 - DNA, 25 bp, synthetic construct
  source 1..25: mol_type = other DNA
  modified_base 1: mod_base = OTHER; note = 2'-O-methyl modified nucleotide
  modified_base 2: mod_base = OTHER; note = 2'-fluoro modified nucleotide
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-fluoro modified nucleotide
  modified_base 7: mod_base = OTHER; note = 2'-O-methyl modified nucleotide
  modified_base 8: mod_base = OTHER; note = 2'-O-methyl modified nucleotide
  modified_base 9: mod_base = OTHER; note = 2'-O-methyl modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-fluoro modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-O-methyl modified nucleotide
  modified_base 16: mod_base = OTHER; note = 2'-fluoro modified nucleotide
  modified_base 17: mod_base = OTHER; note = 2'-O-methyl modified nucleotide
  modified_base 18: mod_base = OTHER; note = 2'-fluoro modified nucleotide
  modified_base 19: mod_base = OTHER; note = 2'-O-methyl modified nucleotide
  modified_base 20: mod_base = OTHER; note = 2'-fluoro modified nucleotide
  modified_base 21: mod_base = OTHER; note = 2'-O-methyl modified nucleotide
  modified_base 22: mod_base = OTHER; note = 2', 2'-difluoro 2'-deoxycytidine
  modified_base 23: mod_base = OTHER; note = 2', 2'-difluoro 2'-deoxycytidine
  misc_feature 1..21: note = RNA
  misc_feature 22..25: note = DNA
tttgtaatcgtcgataccctgcctt

SEQ ID NO: 13 - DNA, 19 bp, synthetic construct
  source 1..19: mol_type = other DNA
cgctctgtcagccttacta

SEQ ID NO: 14 - DNA, 25 bp, synthetic construct
  source 1..25: mol_type = other DNA
gcgtggaaagcgtagacaaggagat

SEQ ID NO: 15 - DNA, 25 bp, synthetic construct
  source 1..25: mol_type = other DNA
ccttgacgatacagctaattcagaa

SEQ ID NO: 16 - DNA, 19 bp, synthetic construct
  source 1..19: mol_type = other DNA
gggatgggtttgtggagtt

SEQ ID NO: 17 - protein, 17 aa, synthetic construct
  REGION 1..17: note = This sequence is one part of a branched amino acid sequence
KHHHKHHHKHHHKHHHK

SEQ ID NO: 18 - protein, 17 aa, synthetic construct
  REGION 1..17: note = This sequence is one part of a branched amino acid sequence
KHHHKHHHNHHHNHHHN

SEQ ID NO: 19 - protein, 4 aa, synthetic construct
HHHK

SEQ ID NO: 20 - protein, 18 aa, synthetic construct
  REGION 1..18: note = This sequence is one part of a branched amino acid sequence
KHHHKHHHKHHHHKHHHK

SEQ ID NO: 21 - protein, 20 aa, synthetic construct
  SITE 18: note = Side chain of lysine linked via an amide linkage to another sequence
  SITE 19: note = Side chain of lysine linked via an amide linkage to another sequence
  SITE 20: note = Side chain of lysine linked via an amide linkage to another sequence
  SITE 20: note = Lysine carboxamide
KHHHKHHHKHHHKHHHKKKK

SEQ ID NO: 22 - protein, 21 aa, synthetic construct
  SITE 19: note = Side chain of lysine linked via an amide linkage to another sequence
  SITE 20: note = Side chain of lysine linked via an amide linkage to another sequence
  SITE 21: note = Side chain of lysine linked via an amide linkage to another sequence
  SITE 21: note = Lysine carboxamide
KHHHKHHHKHHHHKHHHKKKK

(19) United States
(12) Patent Application Publication
(10) Pub. No.: US 2025/0027086 A1
(43) Pub. Date: Jan. 23, 2025

(54) COMPOSITIONS AND METHODS FOR TREATMENT OF CANCERS USING MODIFIED siRNA-GEM AGENTS

(71) Applicant: Sirnaomics, Inc., Germantown, MD (US)

(72) Inventors: David M. EVANS, Germantown, MD (US); Daniel MUTISYA, Germantown, MD (US); Sean LU, Germantown, MD (US); Bhaskar HALAMI, Germantown, MD (US); Vera SIMONENKO, Germantown, MD (US)

(21) Appl. No.: 18/909,519

(22) Filed: Oct. 8, 2024

Related U.S. Application Data
(63) Continuation of application No. PCT/US23/65574, filed on Apr. 10, 2023.
(60) Provisional application No. 63/329,240, filed on Apr. 8, 2022.

(51) Int. Cl. C12N 15/113; A61P 35/00

(52) U.S. Cl. CPC C12N 15/113; A61P 35/00; C12N 2310/14; C12N 2310/315; C12N 2310/321; C12N 2310/322

(57) ABSTRACT

Pharmaceutical compositions are provided comprising gemcitabine (“GEM”)-containing modified RNAi agents such as siRNA molecules, combined with a histidine-lysine copolymer carrier, intended for inhibiting the expression of targeted genes involved in a variety of cancers. Modifications to the siRNA backbone may include, without limitation, 2′-OMe, 2′-Fluoro and phosphorothioate modifications. Methods for treatment of a variety of cancers by administering such pharmaceutical compositions are further provided, including breast, ovarian, and pancreatic cancer in mammals, among others.

### RELATED APPLICATIONS

The present application is a continuation of International Application No. PCT/US2023/65574, filed on Apr. 10, 2023, which claims the benefit of and priority to U.S. Provisional Patent Application No. 63/329,240, filed Apr. 8, 2022, the contents of which are hereby incorporated by reference in their entireties.

### SEQUENCE LISTING

The instant application contains a sequence listing, submitted electronically in XML format, and is hereby incorporated by reference in its entirety. The XML-formatted sequence listing was created on Apr. 7, 2023, is named 4690_0032I_SL and is 178,201 bytes in size.

### FIELD

Gemcitabine-containing modified RNAi agents or complexes for inhibiting the expression of select targeted genes are provided. Methods for treatment of cancers, in which pharmaceutical compositions comprising these RNAi agents and complexes, are further provided for treating a variety of cancers.

### BACKGROUND

Gemcitabine (“GEM”)-containing RNAi agents, e.g., double-stranded siRNA molecules, in combination with a histidine-lysine copolymer carrier have been described previously for use in treatment of cancers. See e.g., WO2021/067825. Targeted genes are ones that drive tumor development but are not critical for normal cell functioning.

siRNAs are double stranded RNA molecules consisting of a sense strand and a complementary antisense strand. These molecules may have modified backbones and may be blunt ended that are, e.g., 19-29 bases long on each strand or they may exhibit two base overhangs (typically dTdT).

Each strand of the siRNA typically is made on a synthesizer by conjugating the next base in the desired sequence to the previous base attached to the growing oligonucleotide. Amidite chemistry and other synthetic approaches are well known in the field. Once synthesized, the two strands are then annealed to each other to form the duplex.

It has been demonstrated that siRNAs against select targets within a cancer cell may be able to reduce expression of a protein encoded by the silenced gene target. Silencing these genes can, in turn, inhibit growth of that cell. If the cell is specifically a diseased cell (e.g., a cancer cell) that the siRNA can access, then the siRNA may act as a therapeutic. Furthermore, in some cases, it has been found that the use of select therapeutics (small molecule inhibitors, monoclonal antibodies, etc.) that are currently the ‘gold standard’ for therapy can be augmented by silencing genes in select pathways.

GEM (2′, 2′-difluoro 2′-deoxycytidine) is a pyrimidine-based nucleoside analogue that, when administered systemically, is taken up by nucleoside transporters, activated by tri-phosphorylation by deoxycytidine kinase and can then be incorporated into either RNA or DNA. It replaces the nucleic acid cytidine during DNA replication (during cell division) and can inhibit tumor growth; new nucleosides cannot be attached to this nucleoside, resulting in apoptosis of the cells (Damaraju et al., Oncogene 22:7524-7536 (2003)).

GEM is the primary therapeutic in treating pancreatic cancer (Burris et al., J Clin Oncol 15:2403-2413 (1997) but is used in the treatment of a number of other cancers including cholangiocarcinoma (Jo and Song, “Chemotherapy of Cholangiocarcinoma: Current Management and Future Directions. Topics in the Surgery of the Biliary Tree”, chapter 3; p 35-52. http://dx.doi.org/10.5772/intechopen.76134, S. Y. (2018)), non-small cell lung cancer (Muggia et al., Expert Opinion on Investigational Drugs, 21:4, 403-408 (2012)), ovarian cancer (Le et al., Gynaecol. Oncol. Res. Pract. 4:16 (2017) and breast cancer (Xie et al., Oncotarget, 9:7148-7161 (2018)). Gemcitabine is taken up by nucleoside transporters, is activated by deoxycytidine kinase, and is incorporated into both RNA and DNA. Inhibition of ribonucleotide reductase and dCMP deaminase enhances its activation, while cytidine deaminase converts gemcitabine to its presumably inactive metabolite 2′,2′-difluorodeoxyuridine, which in its nucleotide form may inhibit thymidylate synthase. Gemcitabine is administered systemically by IV infusion into the patients. Standard administration of gemcitabine is with a 30-min weekly infusion at 1000 mg/m², but is limited in its utility since it exhibits significant toxicity due to its distribution not only to tumor cells but also into normal cells. Furthermore, to see efficacy in some tumor types, the dose needs to be elevated to very high levels, and the therapeutic index for the drug can be very limited.

GEM is widely used in combination, predominantly with a platinum analog. Other alternatives for combinations of gemcitabine in ovarian cancer consist of increasing the inhibition of ribonucleotide reductase with triapine or hydroxyurea. GEM (like 5-FU and other nucleoside analogs) can be chemically synthesized in a manner to allow direct coupling to DNA or RNA bases through traditional synthetic means (manually or using automated instruments).

Various means have been attempted to overcome GEM toxicity. One of the most promising is the use of targeted delivery agents that selectively enrich delivery to the tumor cells while reducing its delivery to normal cells.

An example of genes that augment the action of the compound gemcitabine have included siRNAs targeting RAD17, CHK1, CHK2, ATR, ATM just to name a few (see: Azorsa, J. Transl. Med. 7:43 (2009); Fredebohm (Journal of Cell Science 126:3380-3389 (2013), and Plunkett et al., Semin Oncol 23:3-15 (1996)). These studies as mentioned above have sought to find additional targets within the cell that, when inhibited (by antagonists such as small molecules or antibodies etc.) or silenced (using siRNA or miRNAs), results in a beneficial shift in the dose response curve for the drug to where it exhibits equal efficacy but at lower doses/concentrations. Such a shift in dose response is seen with siRNAs against, e.g., CHK1.

Delivery of these siRNAs to the tumor environment within the animal/human exhibiting the disease can be accomplished using a variety of targeted or non-targeted delivery agents. These delivery vehicles can consist of lipids, modified lipids, peptide delivery vehicles and the like or can even be via direct attachment of a targeting ligand onto a modified (chemically stable) siRNA molecule through modification of the backbone to prevent degradation of the siRNA by nucleases and other enzymes encountered in the circulation.

Recently, GalNAc modified siRNAs have been used to promote delivery of these siRNAs specifically to hepatocytes within the liver. The GalNAc moieties bind with very high affinity to the asialoglycoprotein receptors (ASGPR) present specifically and at high numbers on the hepatocytes. The ASGPRs are believed to be internalized into the cells upon binding and therefore carry the attached siRNA into the cell with them.

Other targeting ligands that can deliver a payload to specific cell types include the GLP1 peptide (binding to the GLP1 receptor on Pancreatic Beta cells), RGD motifs (e.g., cRGD, iRGD that bind α5β3 integrin receptors or peptides derived from the Foot and Mouth virus (binding with nanomolar affinity to α5β6 integrin receptors compared with almost micromolar affinity for α5β3 receptors)), Folate ligands (that bind to folate receptors), transferrin ligands binding to transferrin receptors and EGFR targeting through EGF receptors. Many other examples of targeting moieties show specificity for delivery to distinct cell types.

The disclosed embodiments provide for pharmaceutical compositions and methods of treatment of cancerous tumors involving co-delivery of a modified siRNA with GEM to produce a therapeutic benefit greater than the effect generated from administration of either the siRNA or GEM alone, or administration of an unmodified siRNA with GEM.

### SUMMARY

Pharmaceutical compositions or formulations are provided in the disclosed embodiments, comprising modified, interfering ribonucleic acid (RNAi) molecules combined with one or more gemcitabine molecules in the place of cytidine molecules. Further included in the compositions is a histidine-lysine copolymer carrier such as HKP or HKP(+H). The RNAi molecule may be an siRNA, miRNA, shRNA or other oligonucleotide capable of silencing or inhibiting at least one targeted gene. Modifications to the backbone include, among others, 2′-OMe, 2′-Fluoro and phosphorothioate modification.

The compositions may comprise one or more of the siRNA molecules identified in FIG. 1 as SEQ. ID Nos. 1-10, and 13-14. In some embodiments, the composition may include a combination of any of SEQ. ID Nos. 1-10 together with at least SEQ. ID No. 13, or SEQ. ID No. 14. In some composition embodiments, lipofectamine may be added. In certain other embodiments the siRNA is a double stranded siRNA with a modified backbone and at least one gemcitabine molecule.

Further provided are method embodiments for treating a variety of cancers in mammals. In one embodiment is provided a method of treating a subject having cancer, and the cancer growth is attenuated or inhibited when the subject is administered an effective amount of a pharmaceutical composition comprising a modified siRNA molecule comprising gemcitabine and a histidine-lysine copolymer carrier, where the siRNA is at least one chosen from among the set of SEQ. ID Nos. 1-10 and 13-14. In some embodiments the cancer to be treated is breast cancer; in other embodiments the cancer is colon or ovarian. In still other embodiments the cancer is urinary bladder, lung, pancreatic or gastric cancer. In some embodiments the composition is administered through the intravenous route; in others it is administered intratumorally, while in still other embodiments the composition is administered subcutaneously. In some embodiments for treating cancers, lipofectamine is included in the composition administered.

In certain embodiments, a subject having cancer is administered a composition comprising SEQ. ID Nos. 9-10 and SEQ. ID No. 8, and further comprising HKP(+H) as a carrier. In certain embodiments involving this combination of siRNA sequences, the cancer being treated is breast cancer. In another method of treatment embodiment, the subject is administered a composition comprising SEQ. ID No. 9-10 and SEQ. ID No. 13, and further comprising HKP(+H) as a carrier. In certain embodiments involving this combination of sequences, the cancer being treated is ovarian cancer. The method of treatment of cancers may involve, in some embodiments, intravenous administration, while in other types of cancer it may involve intratumoral administration of the composition. In certain embodiments in which a pharmaceutical composition is intratumorally administered to treat pancreatic cancer in a subject, the composition comprises SEQ. ID No. 7-8. The expression of a targeted gene, tumor volume or cancer cell levels is/are reduced by an amount greater than 20 percent; in other embodiments, the expression of a targeted gene is reduced by more than 30 to 90 percent; in still other treatment method embodiments, the expression of a targeted gene is reduced by more than 90, and up to 100 percent. In certain method of treatment embodiments, the subject is a mammal. In other embodiments, the subject is a human.

### BRIEF DESCRIPTION OF THE FIGURES

FIG. 1 (a)-(b) show (a) the sense and antisense strands of the prChk1-GEM variant sequences and non-silencing siRNA+GEM (SEQ. ID Nos. 1-12, respectively, in order of appearance); and (b) the sequences of a few additional siRNA molecules (not comprising GEM) used in the examples of this disclosure (SEQ. ID Nos. 13 through 16). The differing fonts within the sequences denote the following: Upper case: 2′-OMe; Lower case: 2′-Fluoro; Upper case/underline: 2′-OH; Phos: Phosphate; GEM: Gemcitabine; and dT: deoxythymidine.

FIG. 2 shows the effect on MiaPaCa-2 cell viability of polyGEM variants, demonstrating an increasingly significant effect on IC₅₀ from by PNG6 (0.0.4 nM), STD1 (0.02 nM), and PNG7 (0.006 nM) versus Gemcitabine (8 nM), all in combination with lipofectamine. (X±SD; n=4).

FIG. 3 (a)-(c) show the effect of two variants of PGN6 (DM and BH), with and without lipofectamine (“lipo”), on cell viability at increasing siRNA levels in (a) BXPC3 (WT); (b) MiaPaCa (G12C); and (c) Panc10.05 (G12D) pancreatic cancer cell lines.

FIG. 4 (a)-(b) show the effect of two variants of PGN6 (DM and BH), with and without lipofectamine, on cell viability in (a) H358 (G12C) and (b) H1299 (WT) lung cancer cell lines.

FIG. 5 (a)-(b) show the effect of these variants on cell viability in (a) HT29 (WT) and (b) LS180 (G12D/WT) colon cancer cell lines. Both variants show potency and efficacy against pancreatic, lung and colon cancer cells. In the absence of lipofectamine, the PNG products produce their effects as a result of release of the gemcitabine (GEM) from the structure. The change in IC₅₀ in the absence of lipofectamine is typically 10 to 1000-fold.

FIG. 6 (a)-(f) show the efficacy of STD1, PGN6 and PGN7 in (a) Panc 10.05 (G12D); (b) MiaPaCa (G12C); (c) H1299 (WT); (d) H358 (G12C); (e) HT29 (WT); and (f) LS180 (G12D/WT) pancreatic lung and colon cancer cell lines, compared to non-silencing siRNA used as a control.

FIG. 7 (a)-(e) show the efficacy of PGN7 and the combination of PGN7 and Wee1 #7 siRNA in (a) Panc 10.05 (G12D); (b) MiaPaCa (G12C); (c) H358 (G12C); (d) H1299 (WT); and (e) LS180 (G12D/WT) pancreatic lung and colon cancer cell lines.

FIG. 8 (a)-(e) show the efficacy of the PGN7 variant and its combination with BCLxL in (a) Panc 10.05 (G12D); (b) MiaPaCa (G12C); (c) H358 (G12C); (d) H1299 (WT); and (e) LS180 (G12D/WT) pancreatic and lung, but not colon, cancer cell lines.

FIG. 9 (a)-(d) show the efficacy of the PGN7+NS (i.e., with non-silencing siRNA), BCLxL #5+NS, and the PGN7+BCLxL #5 combination in (a) the HT1376 (400 cells/well) and (b) HTB-9 (800 cells/well) urinary bladder cancer cell lines; (c) the N87 gastric cancer cell line; and (d) the OVCAR-3, ovarian cancer cell line.

FIG. 10 (a)-(d) show the efficacy of the PGN7+NS (i.e., with non-silencing siRNA), Wee1 #7+NS, and the PGN7+Wee1 #7 combination in (a) the HT1376 and (b) HTB-9 urinary bladder cancer cell lines; (c) the N87 gastric cancer cell line; and (d) the OVCAR-3, ovarian cancer cell lines.

FIG. 11 (a)-(d) show the efficacy of PGN7+NS (i.e., with non-silencing siRNA), BCLxL+NS, Wee1 #7+NS, and the combination of PGN7+BCLxL #5, or PGN7+Wee1 #7, in (a) HT1376 and (b) HTB-9 urinary bladder cancer cell lines; (c) N87 gastric cancer cell line; and (d) the OVCAR-3, ovarian cancer cell lines.

FIG. 12 (a)-(b) show the effect of the PolyGEM variant, PGN6 at 2 mg/kg in HKP(+H) on (a) mean (±SE) pancreatic tumor volume and (b) mean(±SD) body weight in treated mice compared to those in the control group receiving non-silencing (NS) siRNA and HKP(+H).

FIG. 13 (a)-(b) show, in (a) triple negative breast cancer (MDA-MB231), and (b) ovarian cancer (OVCAR3) cell lines, the significant reduction in IC₅₀ following administration of combination of PGN7 with BCLxL #5 compared to administration of either PGN7 or BCLxL #5 separately. (a) IC₅₀=1.1 nM; and BCLxL #5+NS, IC₅₀=0.5 nM. (b) IC₅₀=0.06 nM; compared to the effect of PGN7+NS, IC₅₀=1.1 nM; and BCLxL #5+NS, IC₅₀=0.5 nM.

FIG. 14 (a)-(c) show the separate effects of STD1 and PGN7 on (a) tumor weight, (b) body weight, and (c) tumor volume when treated tumor tissue was implanted and grown for 3.5 weeks in mice. Xenograft tumors from all treatment groups were harvested and weighed on Day 25. Mean (±SE, n=8) tumor weights did not differ significantly among with STD 1, PGN 7 and control groups. FIG. 14 (c) shows results of xenograph tumor growth in NU/NU mice after subcutaneously implanting MIA PaCa tumor cells and twice weekly injections of STD1 and PGN7 beginning at Day 0 in separate treatment groups. Tumor volume (mean±SE) was measured as indicated at shown over time and tested with the ANOVA Tukey Test. Only PGN7 significantly reduced tumor volume beginning at 14 days (p<0.02).

### DETAILED DESCRIPTION

The disclosed embodiments are provided for pharmaceutical compositions and methods of treatment using such compositions. Specifically, disclosed embodiments provide an interfering RNA (RNAi) such as a modified siRNA comprising gemcitabine (GEM) that, together with a histidine-lysine copolymer carrier, act to silence the expression of one or more genes of interest.

In the disclosed embodiments the modified siRNA or other nucleic acid molecules target and bind to complementary sequences on target genes to silence them and elicit the desired therapeutic effect. The siRNA or other nucleic acid molecules are formulated together in a nanoparticle formulation with a polypeptide polymer containing at least one histidine residue and at least one lysine residue. More advantageously in some embodiments, the polypeptide polymer is HKP or HKP(+H). Chemical modification of the GEM-containing siRNA backbone (such as those targeting Chk1) increases serum stability over that of unmodified GEM-comprising siRNA backbones.

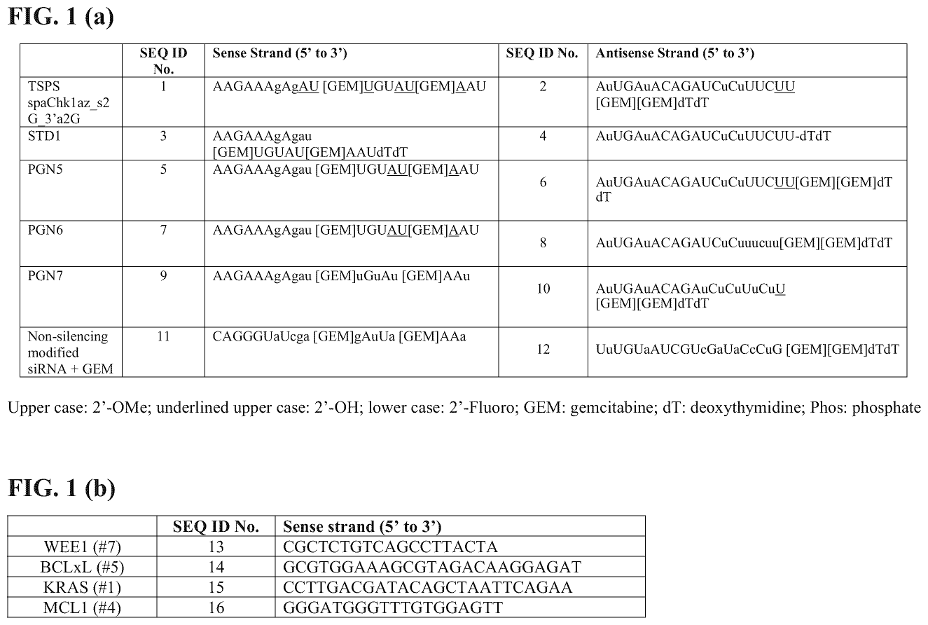

One or more GEM molecules in each siRNA takes the place of one or more cytosine moieties within the modified oligonucleotide (siRNA or other RNAi). Sequences of the sense and antisense strands of the prChk1-GEM variants, as well as the non-silencing siRNA with GEM, are shown in FIG. 1 (a).

### Target Selection for Augmentation of GEM Activity

Constructs containing GEM: Azorsa et al. (J. Transl. Med. 7:43 (2009)) identified siRNAs against the gene CHK1 that showed a potentiation of the effect of gemcitabine in pancreatic cancer cells in culture. Subsequently, Fredebohm (Journal of Cell Science 126:3380-3389, 2013) validated CHK1 as a potentiator of the effect of gemcitabine in pancreatic tumor cells but also identified several other potential targets that improved the activity of gemcitabine when silenced. RAD17 also was identified as one of these targets and demonstrated a profound improvement in the action of gemcitabine in reducing cell viability. This disclosure describes siRNA molecules again CHK targets, but the skilled artisan will recognize that the principles described herein may be applied to GEM-containing siRNA molecules against any target.

CHK1 silencing itself was shown to exhibit some toxicity in cells—even without the presence of gemcitabine—and this would require that the use of an siRNA against CHK1 would need to be administered in a way to enable preferential uptake into tumor cells and not into normal dividing cells.

Polypeptide nanoparticles (“PNP”) prepared using a histidine-lysine branched copolymer can simultaneously deliver multiple siRNAs to the same cell concomitantly. These PNPs also can be used to deliver siRNAs to a wide array of tumor cell types when represented as xenografts in animal models.

As described below, siRNA molecules may be prepared that contain GEM moieties linked to the terminus of the siRNA sequence and/or internal within the sequence. These GEM-containing siRNAs remain functional to silence a target gene and produce enhanced efficacy in killing a tumor via release of GEM into the cell, where a synergistic effect is seen between the gene silencing activity and the effect of the drug, i.e., the result seen is greater than that achieved by administering the siRNA and GEM as separate components individually.

To determine whether GEMs would be better processed if separated by native nucleotides, we took advantage of the fact that gemcitabine is an analog of the nucleotide cytidine and so could be incorporated directly into an siRNA sequence in place of the cytidines (Cs). Gemcitabine amidite may be used to form polymers entirely consisting of GEM moieties. (Ma et al, Chem. Commun., 55:6603-6606 (2019)) These polymers formed nanogels and these demonstrated some activity in cancer cell models in vitro and in vivo. A modified nucleoside incorporating the amidite functional group may be used to introduce the modified nucleoside, such as GEM, into one of the chains in the duplex during their synthesis. Since the antisense strand from the siRNA duplex has to anneal with its appropriate sequence in the sense strand within the siRNA region, and the antisense strand is incorporated into the RISC complex and used to surveil for matching mRNA sequences, the modified nucleosides was first appended to the sense strand of the siRNA to form an extension in length of the sense strand relative to the antisense strand. Multiple (n) non-natural nucleoside bases can be incorporated at the termini of the strand such that delivery of a single siRNA molecule results in co-delivery of multiple (n) non-natural nucleosides.

Once inside the cell cytoplasm, the antisense strand is incorporated into the RISC complex and, if a matching mRNA sequence is identified, the mRNA is cleaved by the enzyme DICER. The SENSE strand is removed during this process and is then cleaved within the cytoplasm. Non-natural nucleoside analogs incorporated onto the ends of the Sense strand will then be cleaved off by endogenous nucleases. The freed molecules of gemcitabine (or similar structures with anti-cancer non-natural nucleoside analogs) then inhibit the tumor cell replication machinery and, in concert with the reduction in the gene expression targeted by the siRNA, will exhibit a better effect on reducing tumor cell growth than in the absence of these agents.

Chemically unmodified siRNA molecules that incorporate GEM residues can be delivered in a suitable nanoparticle formulation that protects the siRNA from degradation in circulation through the blood or in the tissue, tumor microenvironment etc. Once inside the tumor or tissue of interest, the siRNA is released into the tumor cells to produce its cytotoxic effect.

siRNA molecules synthesized using the GEM amidites can be delivered with a histidine-lysine polypeptide (copolymer) nanoparticle (HKP PNP) nanoparticle system. GEMs can be included within an siRNA duplex sequence that targets a gene that then augments the action of the gemcitabine in killing the tumor cells—reducing the amount required for administration to observe a therapeutic effect and decreasing the potential for toxic effects observed when gemcitabine alone is administered in patients. As described below, a previously published siRNA sequence against CHK1 (Azorsa 2009, supra) was selected that had demonstrated potentiation of gemcitabine action when gemcitabine was administered separately from the siRNA (i.e., as individual components).

The siRNAs can be stabilized against nuclease degradation by chemical modification, using methods that are well known in the art, e.g., by use of 2′-OMe and/or 2′-F and/or phosphorothioate modifications. The siRNA can optionally be chemically linked (via the terminus of the SS or AS or even via the terminal GEM added to the molecule) to a targeting moiety (e.g., GalNac, RGD, Folate, TFR, EGFR, peptide targeting ligands, aptamers or other carbohydrates or even small molecules or antibodies or nanobodies). The targeting moiety has affinity for a receptor or other target on the surface of the cells, which enriches uptake into these cells. As with unmodified siRNAs, the SS and AS strands will separate in the cell, the AS strand will result in silencing of the gene of interest while the released SS-polyGEM structure will be degraded, releasing GEM which, in combination with the silenced gene will exhibit greater effect than either alone.

Poly gemcitabine can also be conjugated into a single oligonucleotide, having both sense strand and antisense strands of equal length. After annealing it forms a “poly gemcitabine loop” connected between the two strands of a double stranded RNA. A single oligonucleotide sequence has a reduced cost of manufacture compared to two strands, and also has the potential to have improved stability of the product and the ability to deliver a much larger number of GEM molecules per siRNA delivered into a cell, thereby improving efficacy against the cancer cell. Furthermore, in certain circumstances it may not be necessary for the two strands of the RNA to be of the same length provided there are enough bases in the shorter strand to allow hybridization with the relevant cognate sequence in the longer strand. Under these circumstances the gemcitabine loop may not be symmetrical between the 2 strands.

In another embodiment these constructs are targeted for delivery to specific cell types by attaching a targeting ligand that binds with high affinity to a target present at higher levels on the target cells than on other, non-target, cells. For targeted delivery, multiple ligands can be attached to the gemcitabine loop region—providing the advantage of multivalent targeting by multiple ligands binding to their targets at the same time (increasing avidity of binding and possibly improving cell specificity). The targeting ligands are conjugated with the gemcitabine at the loop region through linkers. This allows delivery of the oligo and gemcitabine conjugates specifically to the targeted cell or tissue, where they can then induce an additive or synergistic or broader therapeutic effect. Ligands may also be added to the Gem loop in the asymmetric strand construct.

Specific examples of CHK1 siRNA molecules containing GEM moieties, as well as the non-silencing siRNAs used in the examples of this disclosure are shown in FIG. 1 (a). FIG. 1 (b) shows additional siRNAs also used in these examples or discussed in this disclosure. The SS and AS strands, in the absence of the GEMs, anneal to form an siRNA targeting CHK1.

Since gemcitabine is a Cytidine analog, it may be used to replace the cytidine moieties in an RNA oligonucleotide sequence with the gemcitabine moieties or the deoxycytidine moieties in a DNA oligonucleotide. These GEMs will still hybridize with their counterparts on a second strand of an oligonucleotide, i.e., GEM will hybridize with a “G” (guanosine/deoxyguanosine) on the opposite strand. Accordingly, the siRNA sequence targeting a gene that, when silenced, produces inhibition of cell growth and can kill tumor cells, is augmented by inclusion of GEM moieties in place of the cytosine moieties in either the sense strand or the antisense strand of an siRNA or an miRNA or even in a single stranded sequence like for an antisense oligonucleotide (ASO). Replacing two cytosine moieties with GEMs in the CHK1_AZ sequence for siRNA silencing the CHK1 gene augments the inhibitory activity of the siRNA alone.

GEM additions can also be on the 3′ or 5′ end of the Sense strand. They also can be added to the same on the AS strand. The additions independently can contain 1, 2, 3, 4, or more GEM moieties.

Other nucleoside anticancer agents can be attached to the oligonucleotide backbone in place of gemcitabine. Such analogs may include Cytarabine (ara-C), 5-FU, 5-Deoxy-5-fluorouridine, Fludarabine, Capecitabine, Cladribine, Troxacitabine or Clofarabine, Azacytidine and 5-deoxy Azacytidine to name just a few examples (see Damaraju, supra).

A number of siRNA sequences can be attached to the polynucleoside analog chains on the sense strand or antisense strand. Examples of such siRNA sequences are siRNAs that silence genes encoding proteins that are counterproductive to the inhibitory mechanism of the nucleoside analogs. For example, an siRNA can be used to silence the enzymes responsible for deamination and inactivation of gemcitabine (Cytidine deaminase or 5′-nucleotidase) or those responsible for enhanced release of the nucleoside from the cell—e.g., nucleoside transporters (see Damaraju supra and references therein).

Other potential targets that could be used as the siRNA sequence in these constructs (attached to Nucleoside analog chains) would include Multidrug resistance (MDR) proteins such as P-glycoprotein (P-gp) and multidrug resistance-associated protein-1 (MRP1), encoded by the MDR1 and MRP1 genes, respectively. Expression of these proteins confers a resistant phenotype to a broad spectrum of drugs used in cancer chemotherapy. These transporters are broadly classified as ATP-binding cassette proteins (ABC) and constitute a superfamily of proteins. Silencing of the MDR1 and MRP1 genes using siRNA has been described. See Dönmez and Gündüz, Biomed Pharmacother. 65(2):85-9 (2011). Other examples of genes that can be silenced include methyl transferases, organic anion and cation transporters (OAT and OCT), oxidoreductase flavoproteins and Nucleoside Transporters (NTs), all of which are involved in drug transport and metabolism in cells and may impart drug resistance phenotypes to cancer cells.

Other gene targets for the siRNAs are those that have been validated to augment the action of gemcitabine or the other nucleoside analogs when silenced themselves. Such gene targets include ATR, RAD17, and WEE1, in addition to CHK1. As other targets are silenced and demonstrated to augment nucleoside activity, these can also have siRNAs against them coupled to the appropriate nucleoside analog.

In using the Azorsa 2009 sequence there were 2 Cs that could be replaced on the SS and on the AS strands respectively. We therefore made a construct where just the SS was modified with incorporation of 2 Gems in place of Cs and we made the AS strand in the same way. We then annealed the SS containing 2 GEMs with an unmodified AS strand and we annealed a GEM-modified AS strand with an unmodified SS. We further annealed the GEM modified SS with the GEM modified AS strand.

The siRNA sequence identified by Azorsa et al. 2009 (CHK1_AZ) is an example of an siRNA that can be used to prepare molecules containing GEM moieties. Using an oligonucleotide synthesizer, the following siRNA strands were manufactured with and without GEM residues included in place of the natural cytidine (“C”) groups present in the sequence.

SiRNA synthesis with GEM

The CHK-Az siRNAs were 19-mer siRNAs containing a 2 base (dTdT) overhang on the 3′ end of each strand. When modified with the inclusion of the gemcitabine moieties within the Sense strand, we inserted these between the end of the sense strand and before the dTdT end groups. The dTdT ends may help stabilize the siRNA sequences against nuclease degradation and this, in turn, may affect the rate of release of the Gems from the Sense strand when it is separated from the AS in the RISC complex during surveillance of the AS strand for the cognate mRNA sequence to be cleaved and silenced.

Analogs of siRNA molecules are synthesized where 2, 4 or 6 GEMs are appended on the 3′ end of the SS and also designed several different siRNAs where the Cs within the sequence could be modified by inserting GEM in their place. GEMs were inserted in place of Cs within the Sense Strand and in the Antisense Strand separately or in combination to explore whether increasing GEM content improved efficacy or potency. Unmodified CHK1 siRNA, C1D-2A, C1D-2S and ClD-4 were transfected into the pancreatic cancer cell line BxPC3 that has been shown to be sensitive to CHK1+Gem (Azorsa 2009, supra). In previous work, a dose response of gemcitabine alone was carried out, together with transfection of non-silencing siRNA (as a control).

It was observed previously that appending additional GEMs to the 3′ end of the SS (ahead of the usual dTdT end groups) had no additive effect greater than adding 2 GEMs and there was no increase in potency and efficacy when 4 or 6 were added. It was suspected that the nucleases present within a cell are unable to cleave the polyGEM sequences between the GEM moieties but can only cleave a GEM from its prior unmodified nucleotide—releasing just 1 GEM from within the polyGEM sequences irrespective of the number of GEMs in the sequence. Consequently, constructs with 4 or 6 GEMs at the 3′ end of the SS had equal or slightly worse potency when compared with 2GEMs at this location.

Rationale for Improvement with CHK1-Targeted siRNAs with Modified Backbones and GEMs

The 25mer siRNAs we designed were blunt ended siRNAs whereas the siRNA sequence used by Azorsa 2009 was a 19mer siRNA with a 2 base (dTdT) overhang at the 3′ end. When two GEM moieties were added between the last nucleotide in the siRNA SS and the dTdT overhang we saw that this sequence showed much higher efficacy against MiaPaca cells than BxPC3 cells. The inclusion of the dTdT on the end of the sequence may decrease the rate of release of the GEMs from the SS in this construct when the SS is unwound from the AS strand and released into the cytoplasm. This may allow the AS sequence to induce silencing of the gene before the GEMs are released from the SS and this may augment the efficacy of this combination in MiaPaca cells compared with BxPC3 cells.

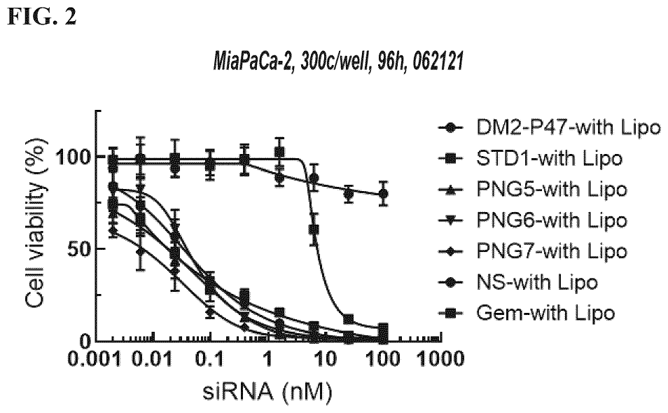

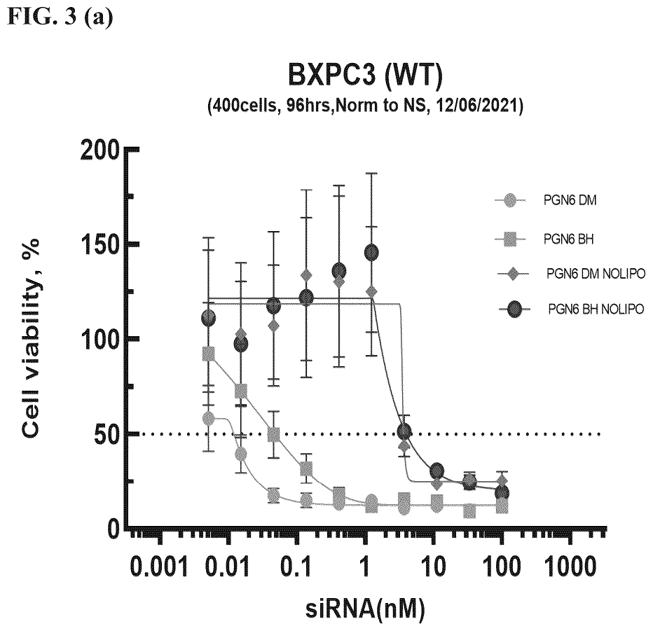

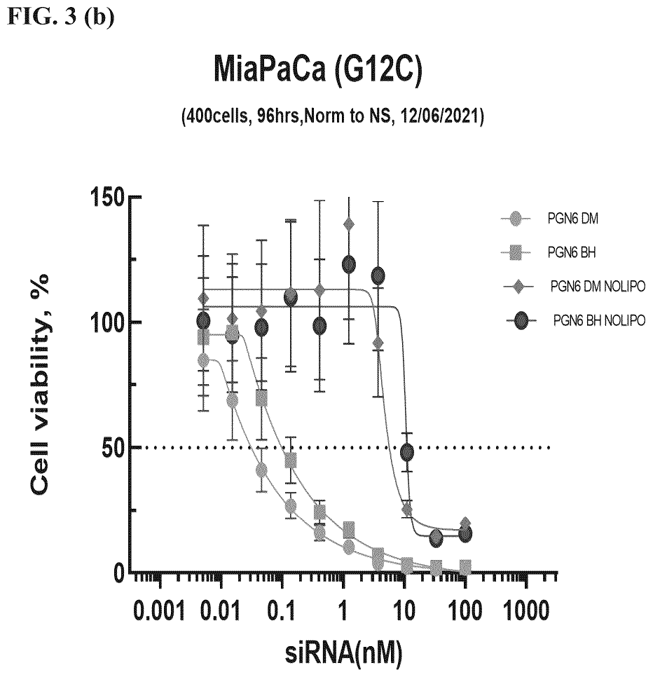

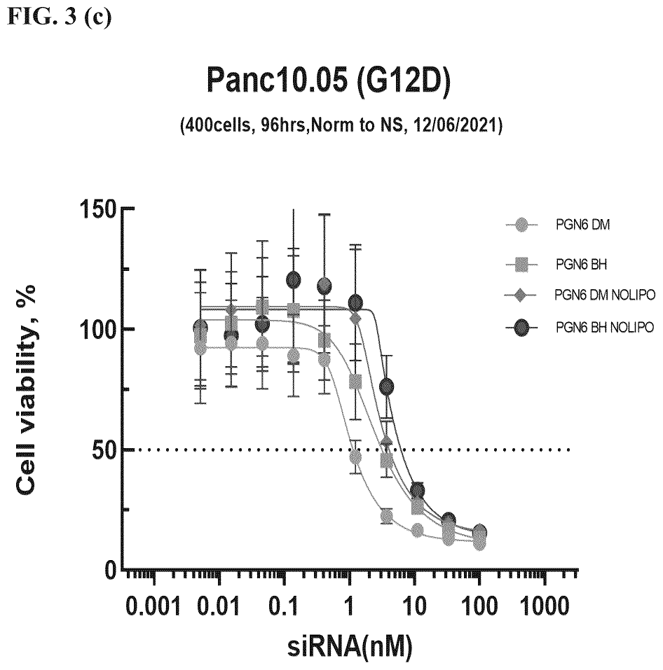

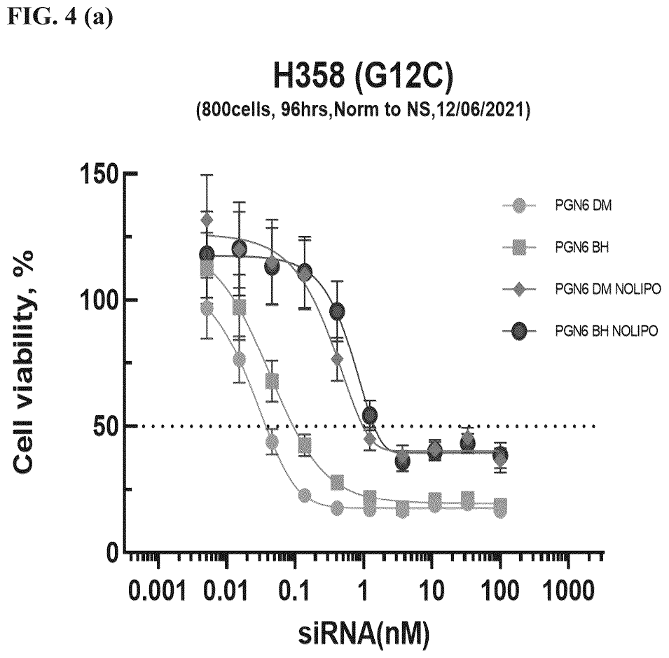

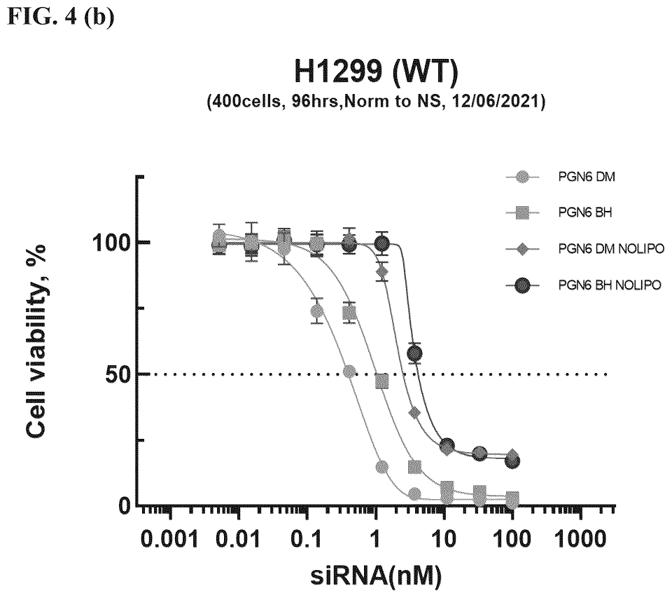

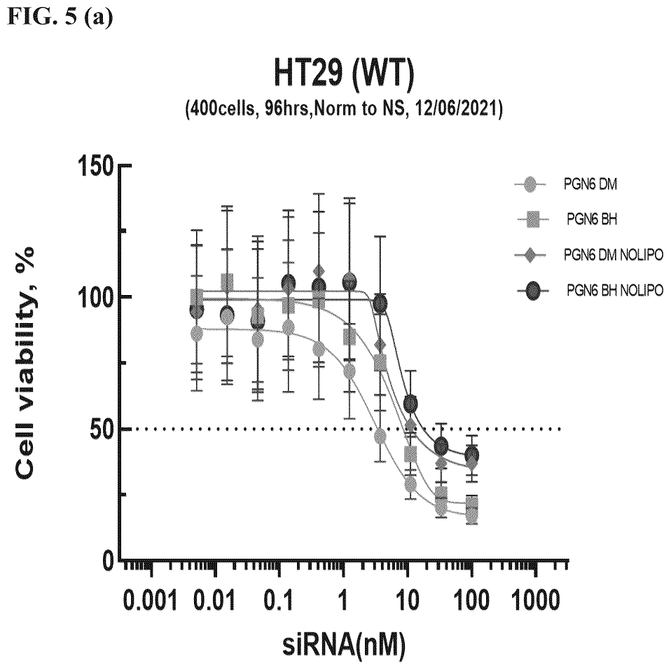

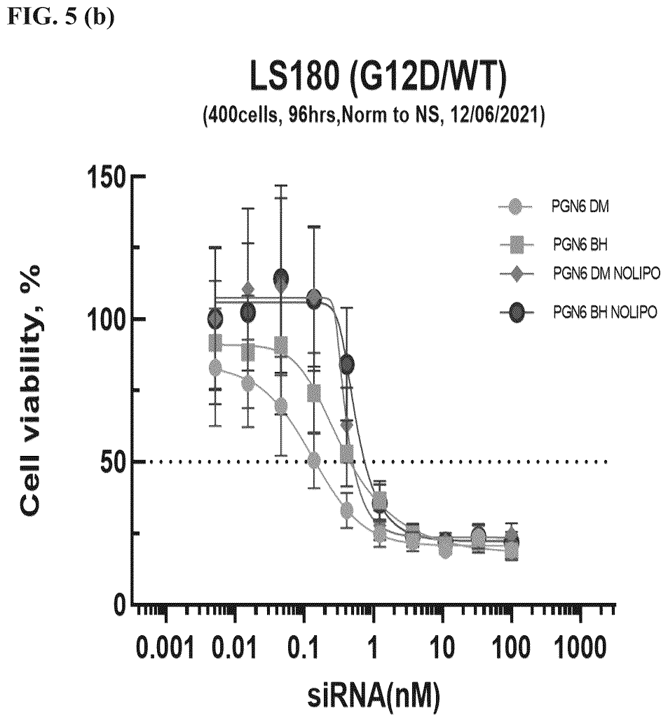

As discussed further in the examples below, in MiaPaCa cells, the modified-backbone PGN7 siRNA (SEQ. ID No. 7-8), was more effective with lipofectamine (ID₅₀0.006 nM) than other siRNAs with lipofectamine: PGN6 (ID₅₀ 0.04 nM), STD1 (ID₅₀0.02 nM) and in contrast to GEM alone (ID₅₀=8 nM) (FIG. 2A-2B). When PGN6 siRNA were administered with lipofectamine, it was more effective than PGN6 without lipofectamine in not just MiaPaCa (G12C) cells, but BXPC3 (WT) cells and Panc10.05 (G12C) cell lines in pancreatic tissue (FIG. 3A-3B). Similarly, in both lung (FIG. 4A-4B) and colon (FIG. 5A-5B) tissue, PGN6 with lipofectamine was apparently more effective in a number of cancer cell lines. In the absence of lipofectamine, PGN6 produces its effect due to the release of the GEM from the nucleic acid sequence. The change in IC₅₀ in the absence of lipofectamine compared to with it typically ranges from 10 to 1000 fold (cf. FIG. 3A-3B).

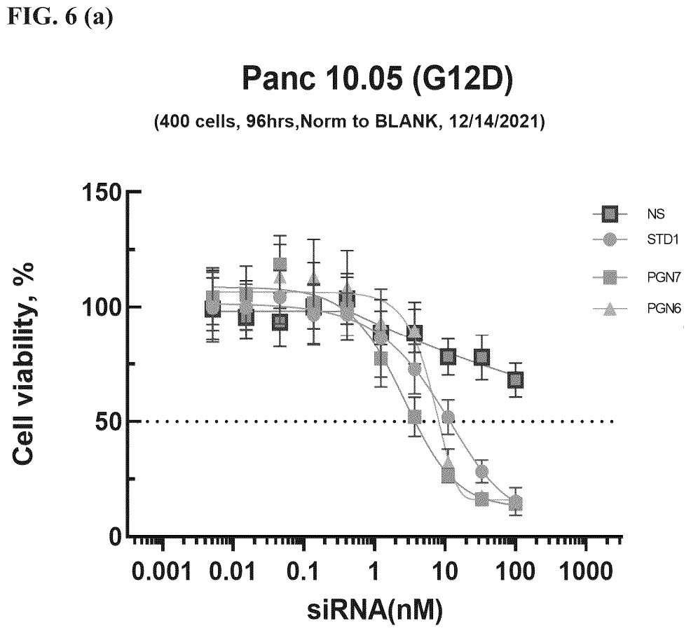

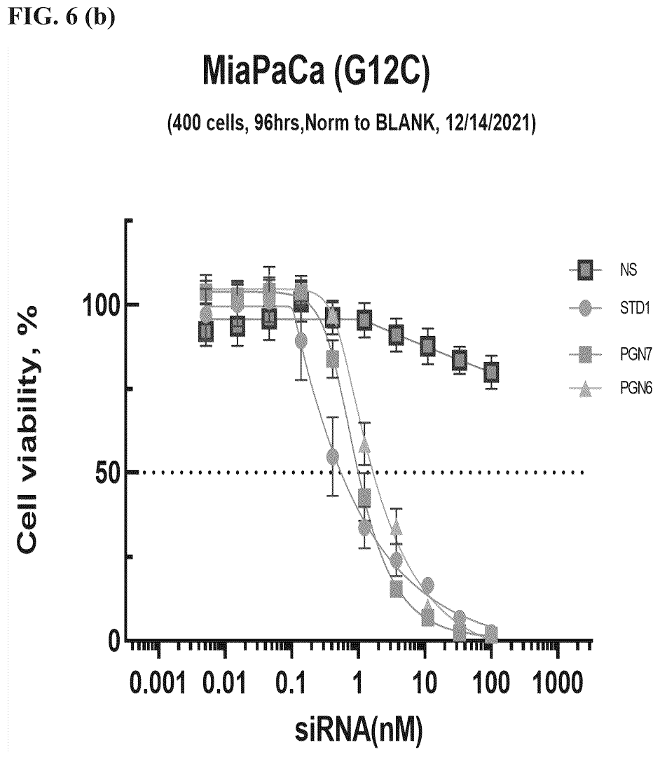

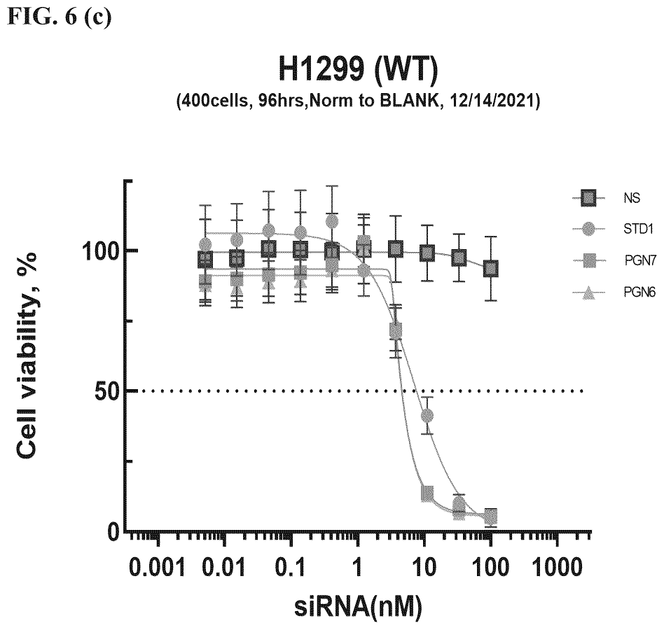

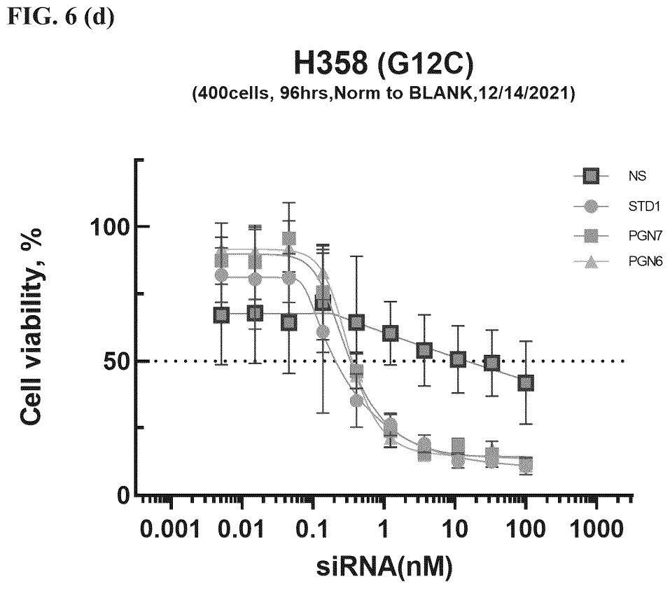

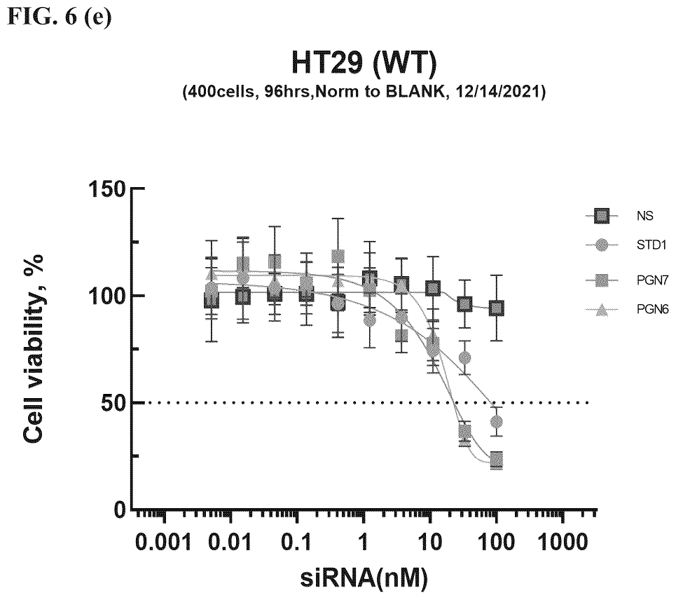

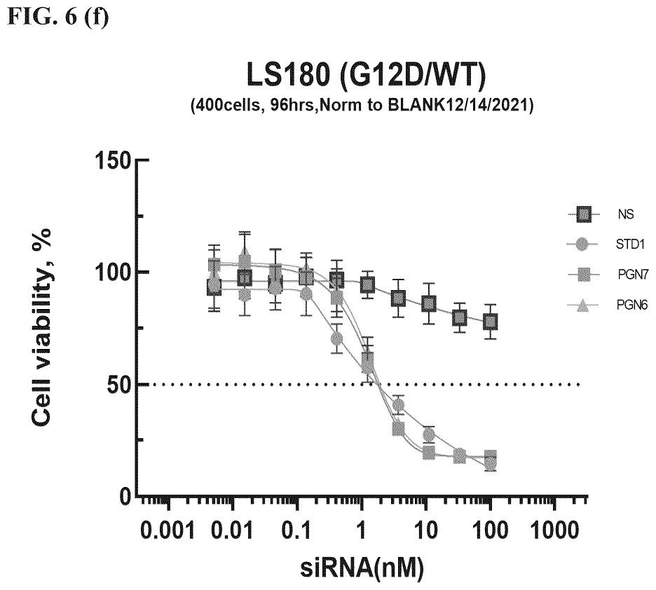

When the STD1 (SEQ. ID No. 3-4), PGN6 (SEQ. ID No. 7-8) and PGN7 (SEQ. ID No. 9-10) siRNAs were evaluated in pancreatic (FIG. 6 (a)-(b)), lung (FIG. 6 (c)-(d)) and colon (FIG. 6 (e)-(f)) tissue against a non-silencing agent (as a control), all three modified siRNAs alone were effective in reducing cell viability.

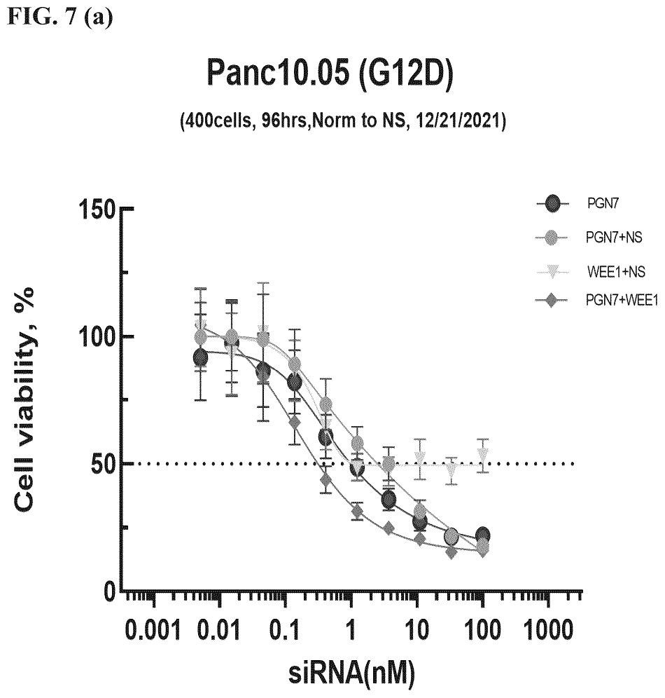

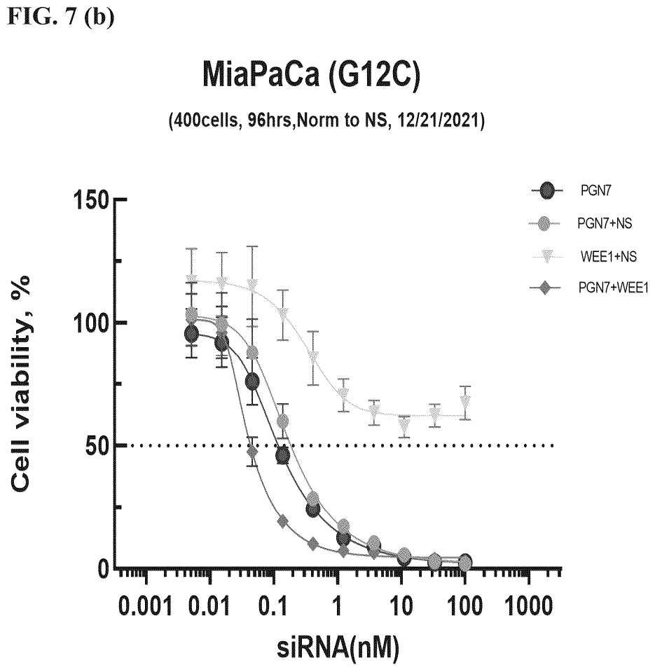

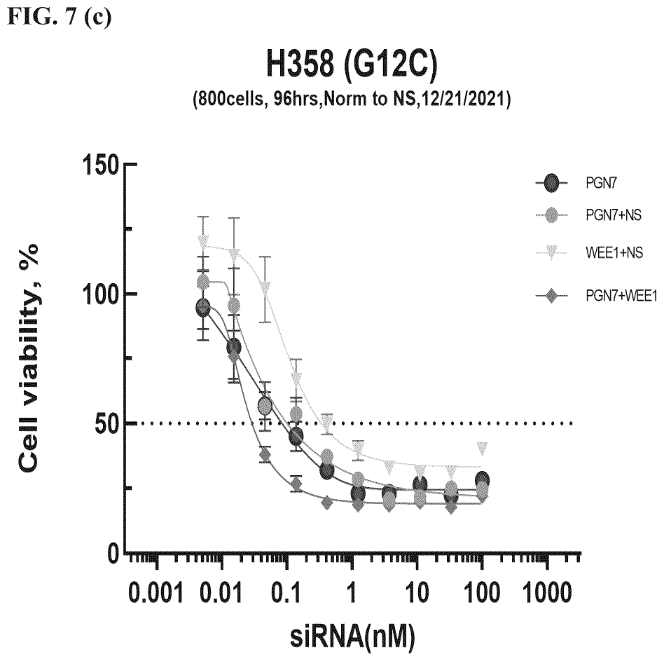

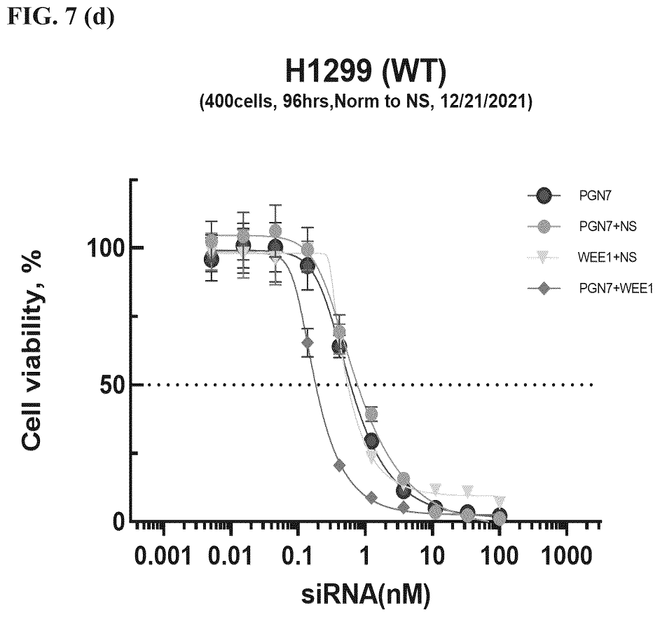

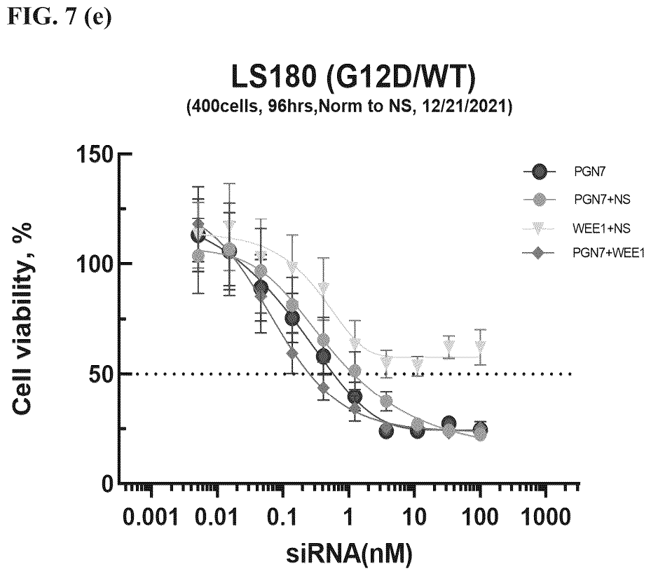

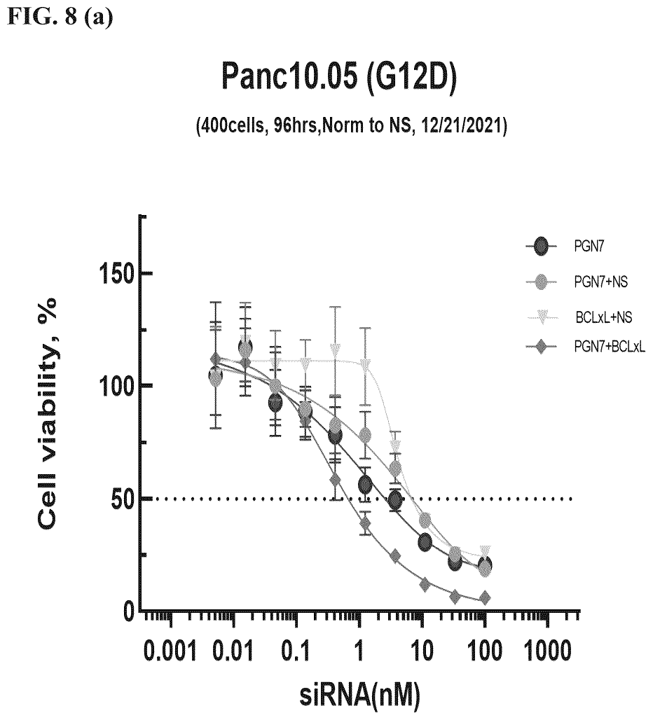

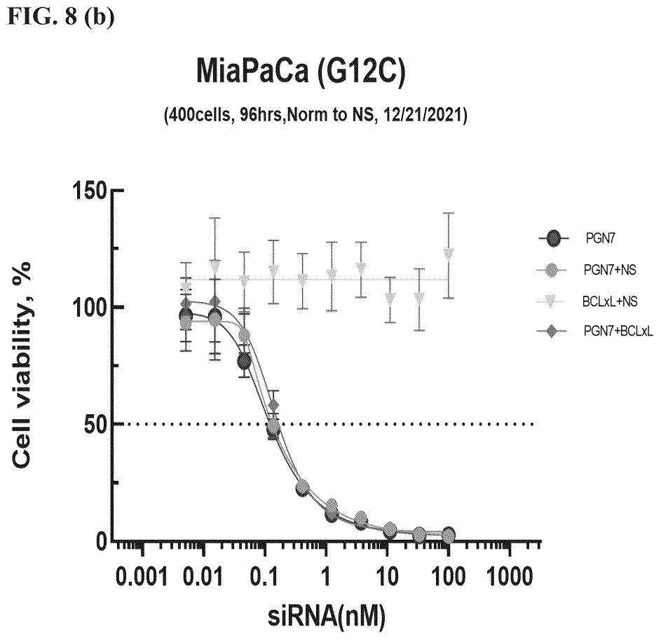

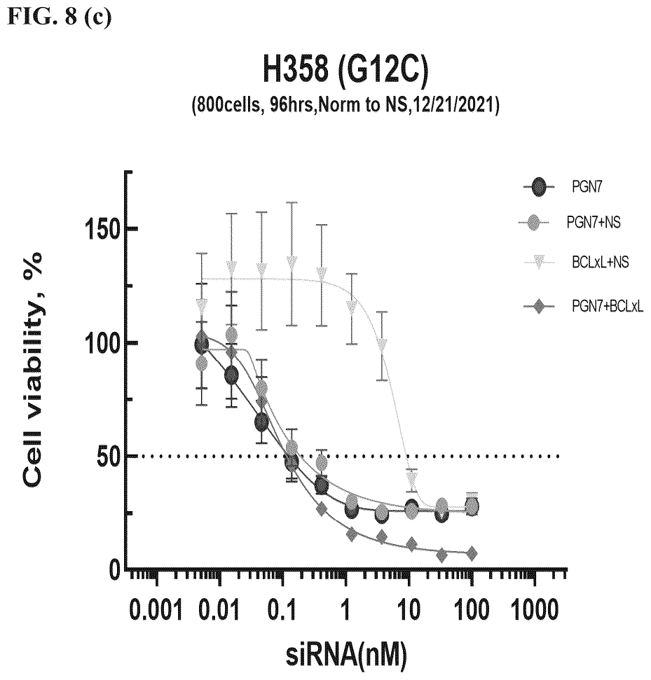

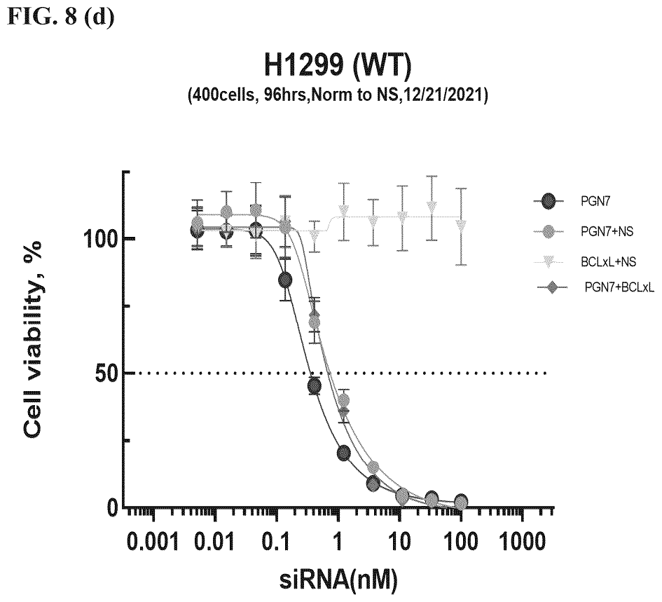

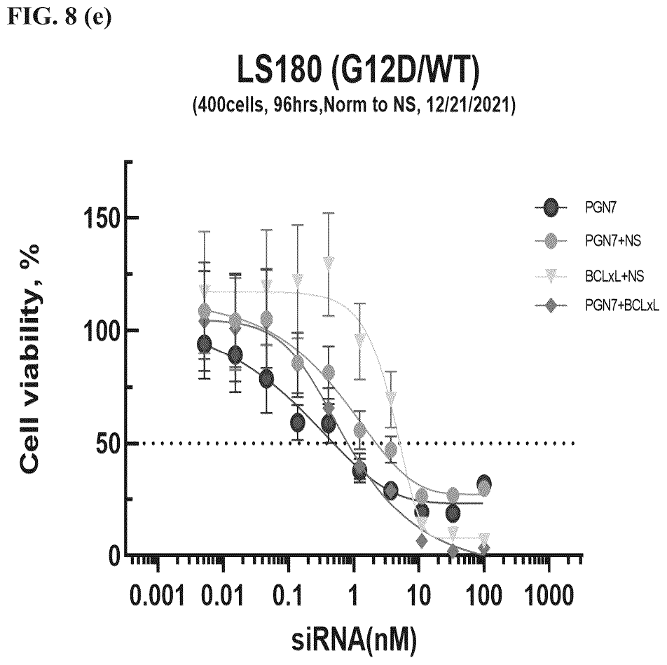

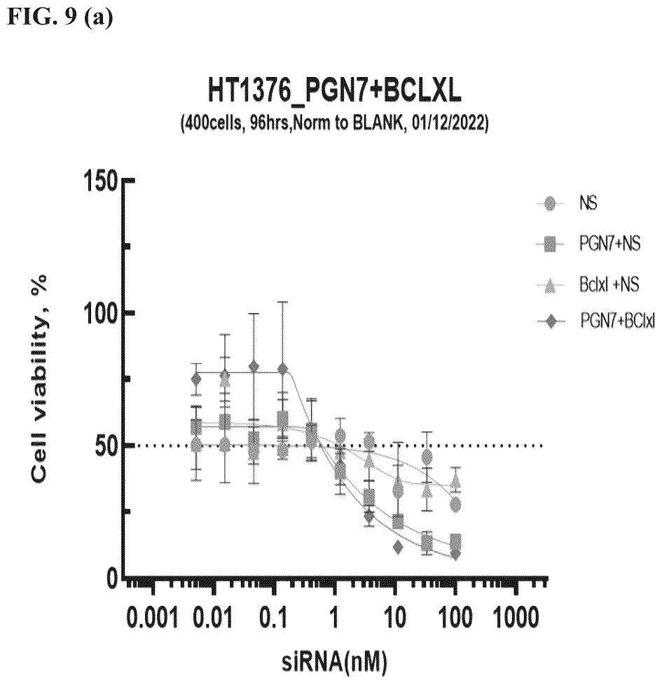

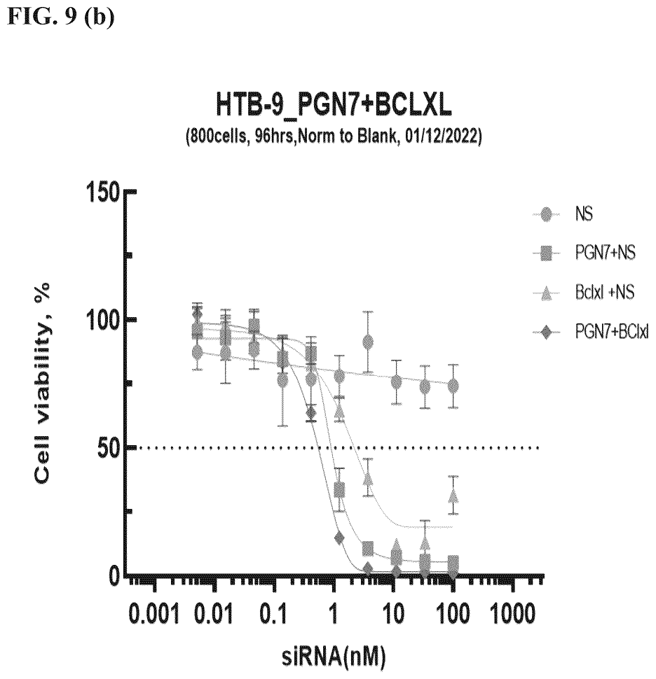

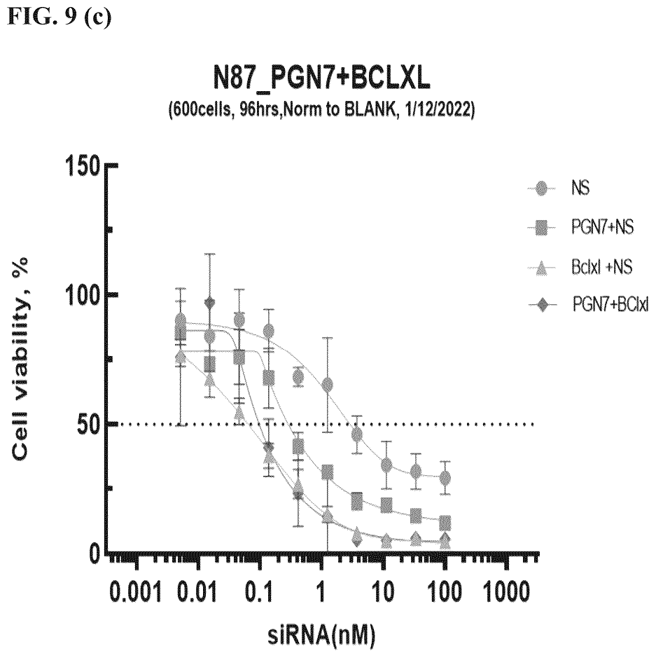

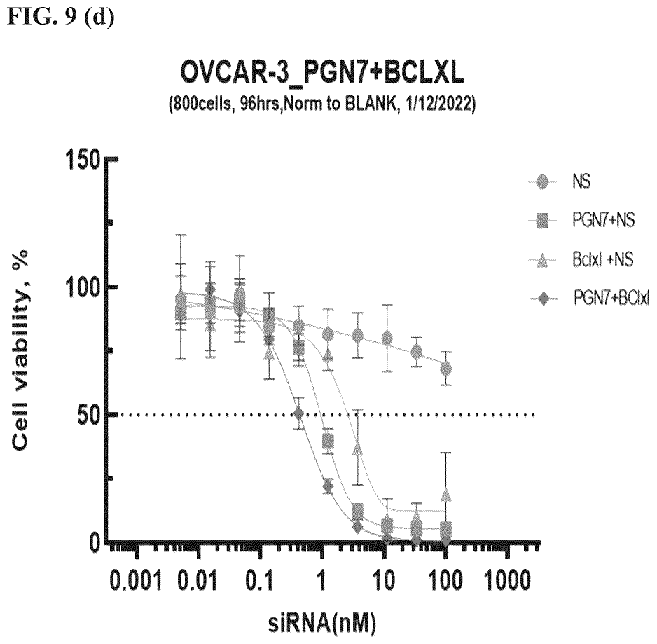

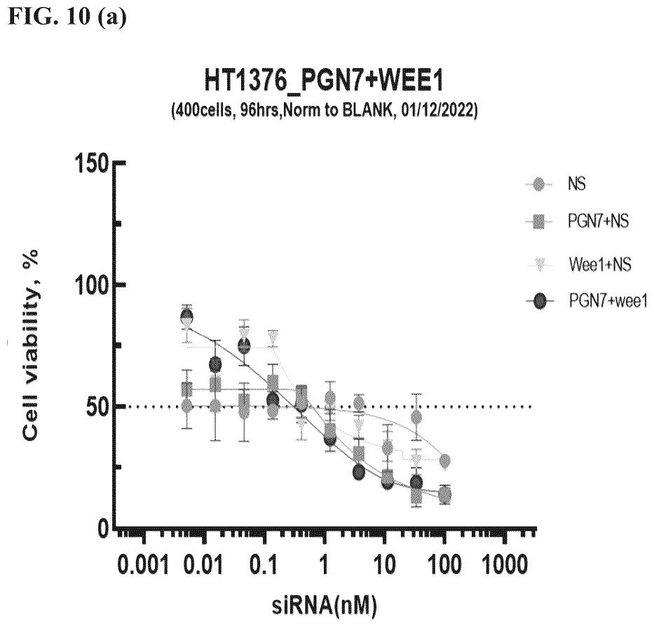

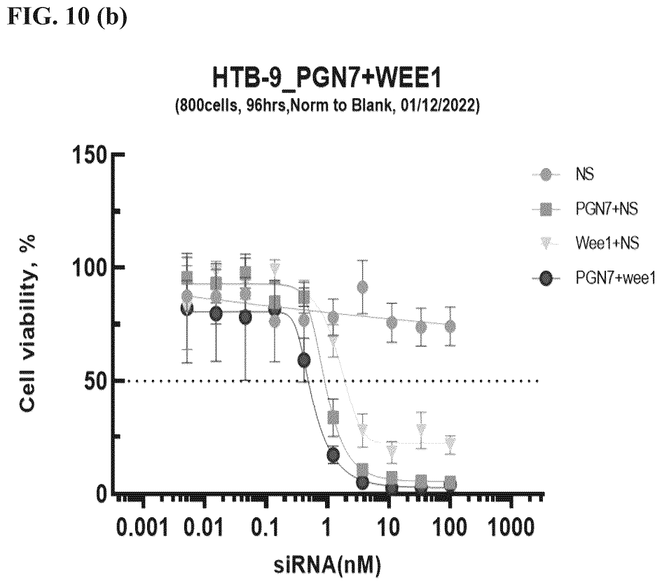

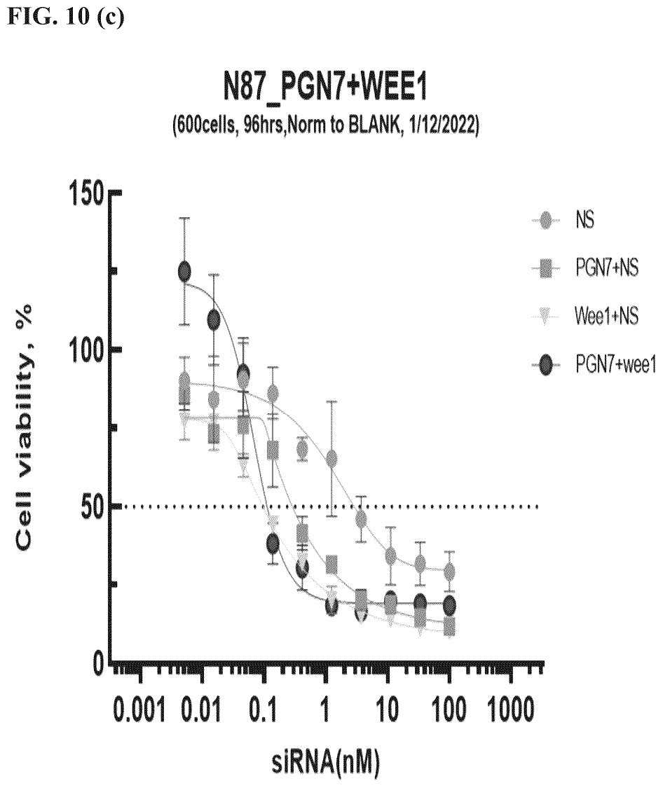

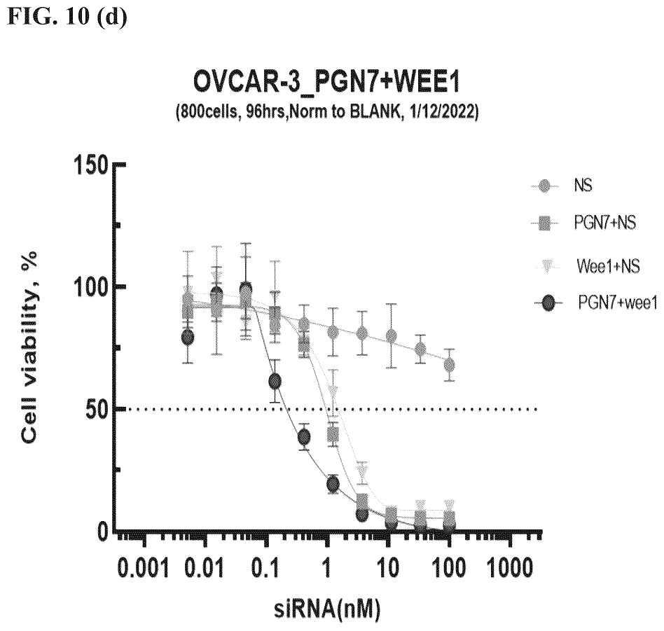

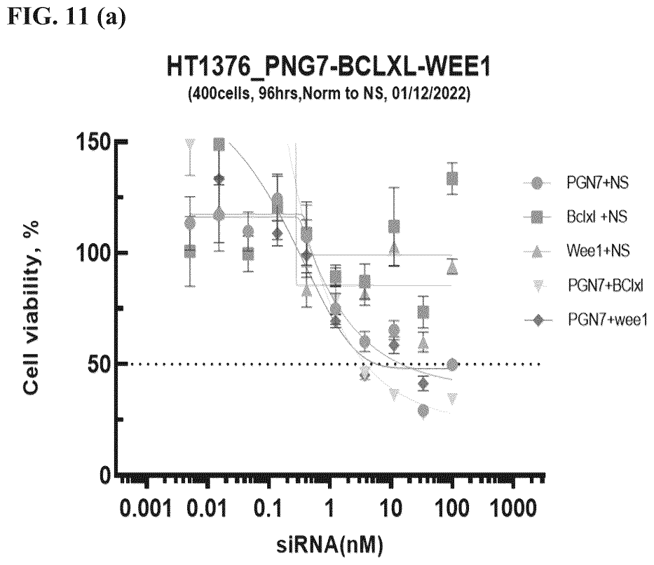

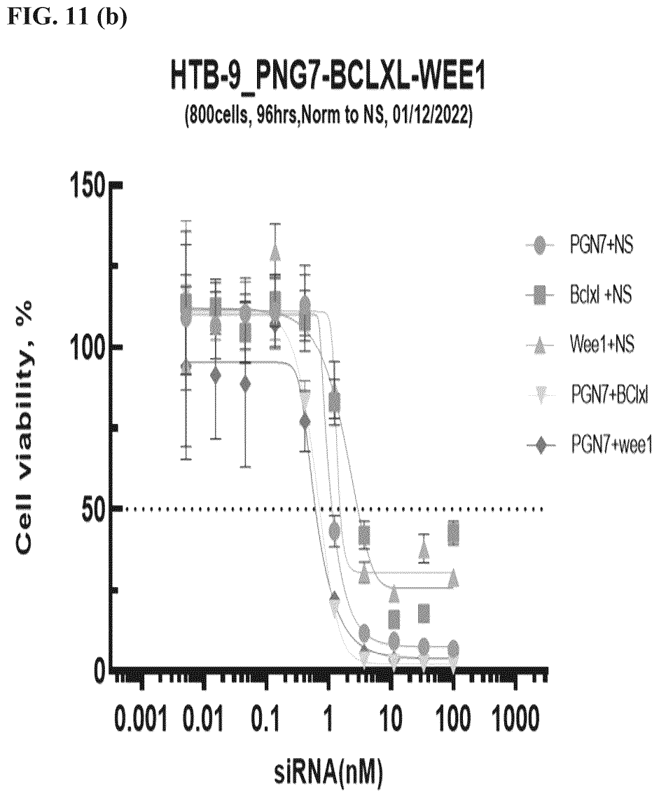

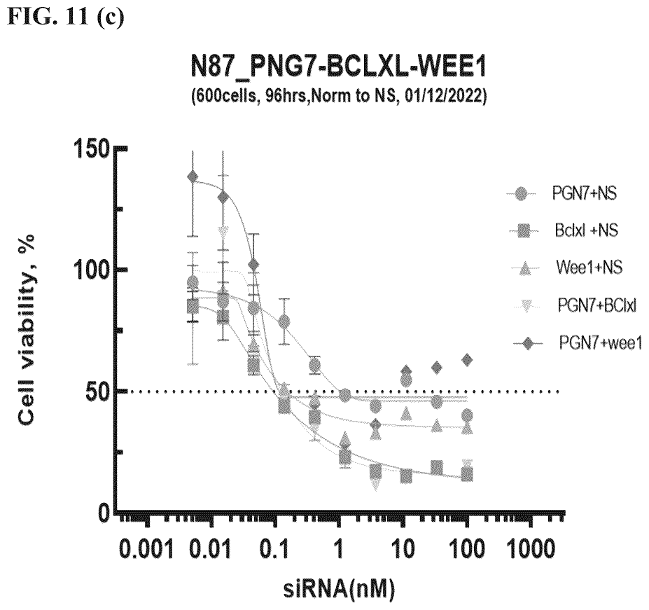

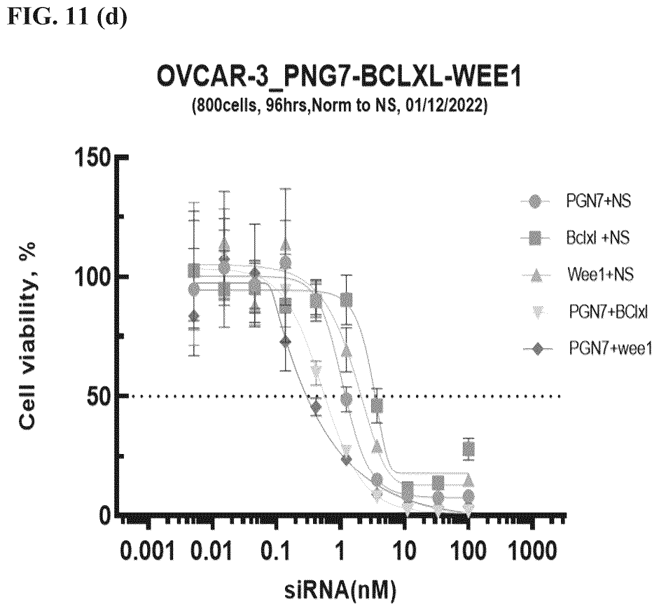

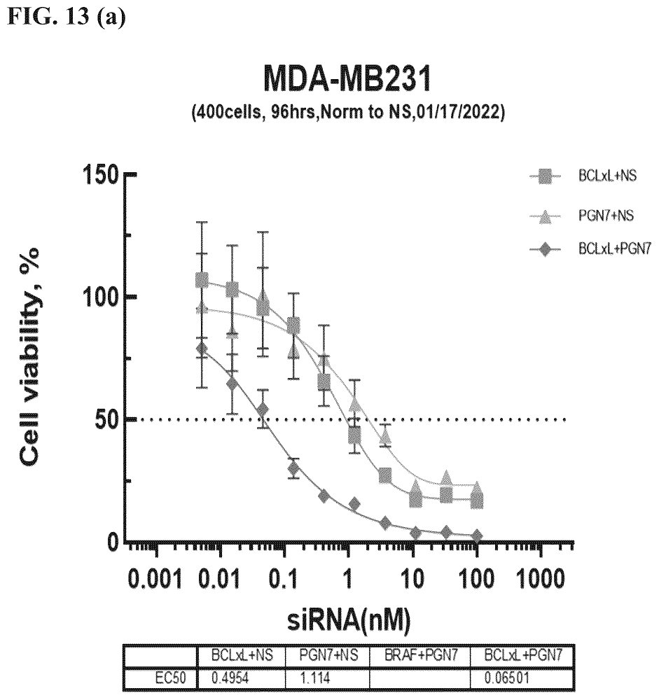

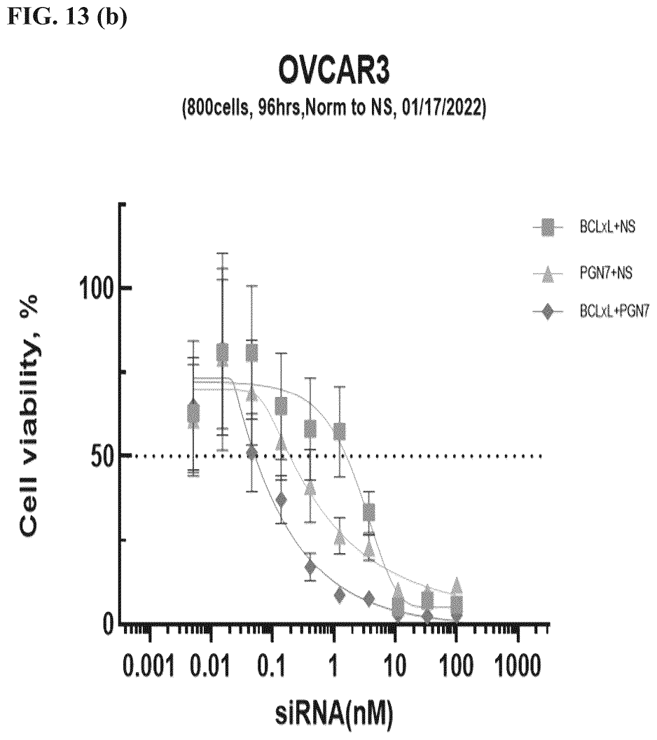

The combination of PGN7 with either WEE1 #7 or BCLxL #5 were surprisingly and consistently effective in lowering IC₅₀ in a variety of tissues and cancer cell lines as well as or better compared to the individual siRNAs. siRNAs PGN7 (SEQ. ID No. 9-10) and WEE1 #7 (SEQ. ID No. 13) were evaluated alone and in combination in the same three tissues (FIG. 7). In each of pancreatic, lung and colon tissues, the combination of PGN7 and WEE1 #7 as compared to each siRNA individually was more effective in a number of cancer cell types. Likewise, the same phenomenon was observed with the combination of PGN7 and BCLxL #5 was more effective than either siRNA individually in the same tissues and cancer cell types (FIG. 8), as well as in three additional tissues (urinary bladder, gastric and ovarian cancer tissues) (FIGS. 9-10). When combinations with PGN7 were compared in the same study, each of PGN7+BCLxL #5, and PGN7+WEE1 #7 was more effective than any individual siRNAs (FIGS. 11 and 13). In contrast, combinations of the modified siRNAs with GEM with each of the siRNAs against KRAS (SEQ. ID No. 15) and MCL1 (SEQ ID No. 16) were tested, but neither showed efficacy (data not shown), emphasizing the specificity shown by PGN7 (SEQ. ID Nos. 9-10)/BCLxL #5 (SEQ. ID No. 14) and PGN7/Wee1 #7 (SEQ. ID No. 13) combinations in these examples.

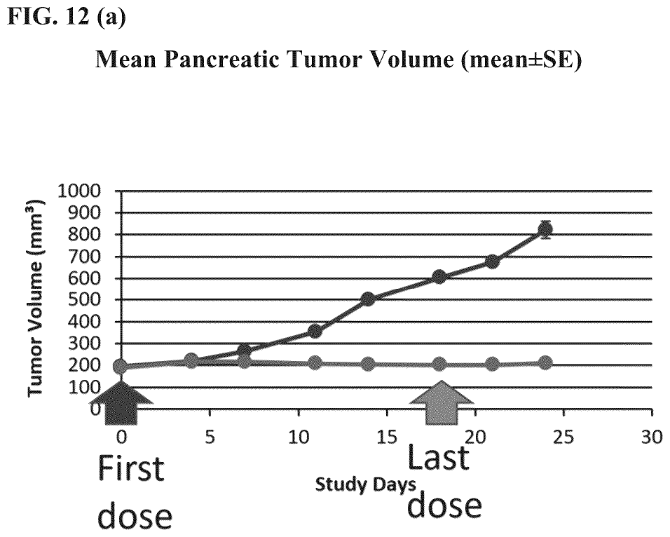

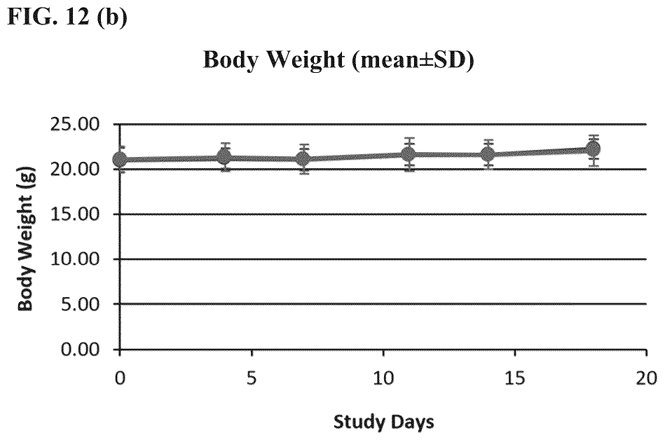

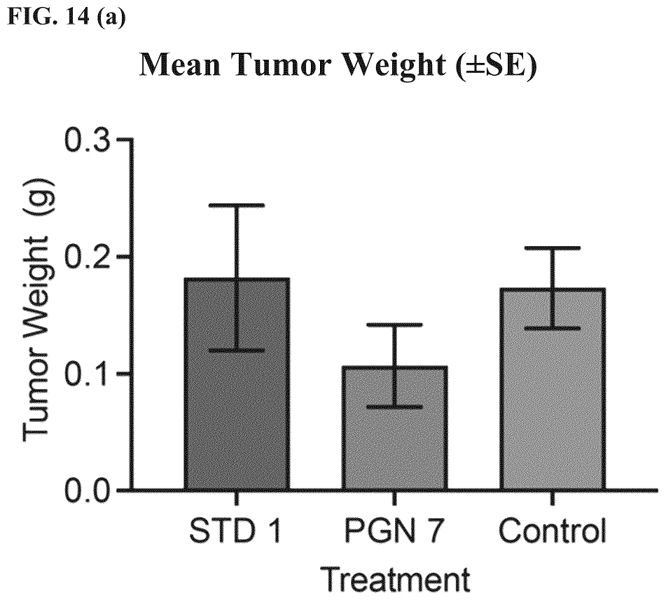

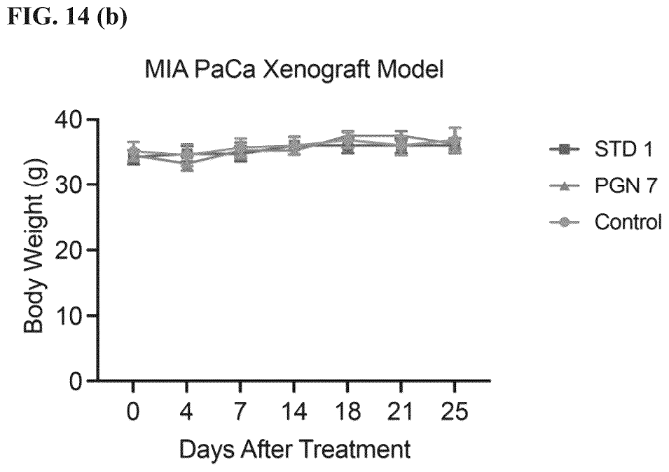

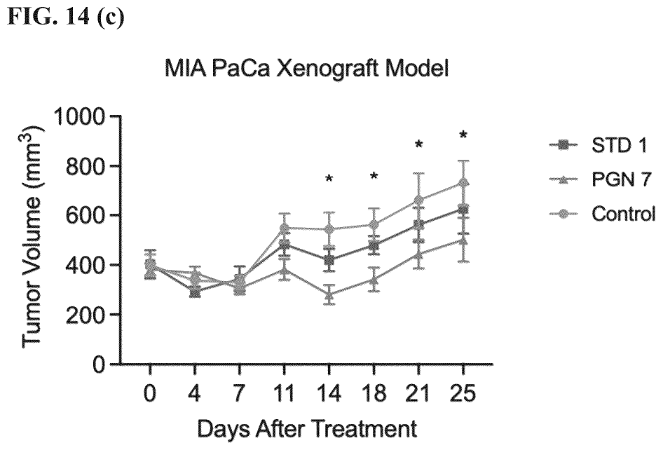

In vivo work to-date has had similarly led to positive results with PGN6 (SEQ. ID Nos. 7-8) and PGN7 (SEQ. ID Nos. 9-10), attenuating the growth of pancreatic tumors in mice (FIGS. 12 and 14).

siRNA Against BCLxL

Proteins in the BCL-2 family are regulators of the intrinsic apoptosis pathway. They contain one to four BCL-2 homology motifs (BH1-BH4) and can be divided into pro-apoptotic and antiapoptotic proteins. The anti-apoptotic multidomain members (BH1-BH4) include BCL-2, BCLxL, BCL-w, BFL-1/A1 and MCL-1, and these proteins function to counteract the pore-forming activity of the pro-apoptotic multidomain proteins BAX and BAK which permeabilize the mitochondrial outer membrane. Following various stress signals, the BH3-only proteins either neutralize the anti-apoptotic proteins or directly activate effector proteins BAX and BAK which will eventually lead to apoptosis in cells. Campbell and Tait, Open Biol. 8: 180002 (2018); Quayle et al., Oncotarget 8:88670-88688 (2017). Cancer cells can evade apoptosis, triggered by oncogenesis or drug treatment by overexpressing the BCL-2 antiapoptotic proteins. Hanahan and Weinberg, Cell 144:646-674 (2011). The siRNA sequence used to target BCLxL #5 is shown in FIG. 1B as SEQ. ID No. 14.

siRNA Against WEE1

WEE1 #7 is a tyrosine kinase in the Ser/Thr family of kinases that plays an important role in the timing of cell division and DNA replication and, as a result, has been seen as a natural target in cancer therapies (Do, K., et al. Cell Cycle 12(19):3159-3164 (2013)). The siRNA sequence used to target WEE1 is shown in FIG. 1B as SEQ. ID No. 13.

siRNA Against KRAS

The KRAS signaling protein plays an important role in relaying instructions for cell growth and differentiation. Mutations in the KRAS gene are frequently detected in pancreatic and colon cancers. The siRNA sequence used to target KRAS #1 is shown in FIG. 1B as SEQ. ID No. 15.

siRNA Against MCL1

Small molecular inhibitors developed against both BCLxL (ABT-100/ventoclax; Souers et al Nat. Med. 19:202-208 (2013)) and MCL1 (563845; Li et al., Leukemia 33L:262-266 (2019)) have shown therapeutic benefit in treating a number of cancer types including cervical cancer (Rahman et al., Biochem. Biophys. Reports, 22: 100756 (2020)), and lung squamous cell cancer (Clare et al., Oncogene 37:4475-4488 (2018)), and head/neck cancers (Thomas et al., Oncotarget, 10:494-510 (2019)). It has also been shown that BCLxL and MCL1 siRNAs can inhibit ovarian tumor growth (Brotin et al., Int. J. Cancer 126:885-895 (2010)). The siRNA sequence used to target MCL1 #4 is shown in FIG. 1B as SEQ. ID No. 16.

### Improved Options for Cancer Therapy

From the data presented in the examples we have demonstrated a dramatic improvement in the efficacy of GEM action in a variety of types of cancer cells by including gemcitabine with one or more modified siRNA. Incorporation of multiple GEMs into the sense strand of an siRNA can deliver GEMs to the same cell where the siRNA is having its effect. In previous studies (Azorsa 2009) it was shown that silencing CHK1 could augment the action of gemcitabine. Therefore, adding GEMs to the siRNA sequence able to silence CHK1 we would expect some additivity/synergy in the interactions. Using a Histidine Lysine polypeptide nanoparticle (PNP) we have demonstrated the ability to deliver siRNAs to many different tumor types in vitro and in vivo. We therefore are studying the delivery of the GEM CHK1 siRNAs as a new therapeutic modality to treat cancer. Furthermore, a polypeptide nanoparticle is able to deliver more than one siRNA against more than one target to the same cell at the same time so we can also silence a second gene target that can further synergize with the first siRNA to maximize the effect in the target cell.

### Gemcitabine Options

Gemcitabine activity can be augmented by inhibition of ribonucleotide reductase and dCMP deaminase.

Gemcitabine is widely used in combination, predominantly with a platinum analog, with other combinations less frequently used or currently being explored. Standard administration of gemcitabine is with a 30-min weekly infusion at 1000 mg/m², but alternatives are being explored such as prodrugs (e.g., CO-1.01, which can bypass transport deficiency), the fixed-dose rate infusion (10 mg/m²/min), and local routes of administration by a 24-h hepatic artery infusion, by instillation in the bladder or by intraperitoneal administration to treat advanced ovarian cancer. Other alternatives for combinations of gemcitabine in ovarian cancer consist of increasing the inhibition of ribonucleotide reductase with triapine or hydroxyurea. Gemcitabine's action on signaling also provides a rational concept for combination with signal transduction pathways.

Gemcitabine as an amidite can be included during synthesis of oligonucleotides and incorporates into the nucleotide sequence. We have previously demonstrated that addition of GEMs to an siRNA can allow co-delivery of the siRNA and the gemcitabine to the same cell. By selecting an siRNA against a molecular target that is expected to improve the effect of gemcitabine in reducing the viability of tumor cells we can see pronounced improvements in efficacy not seen with either agent alone.

While we elected to use siRNAs targeting CHK1, the gemcitabines could be incorporated into other oligonucleotides with similar results. Examples of siRNAs that augment gemcitabine activity have also described cMyc as a suitable gene target (Zhang et al., Cancer Metastasis Rev 26:85-110 (2013)). So an siRNA against cMyc could also have GEM incorporated directly and used to treat cancer, e.g., NSCLC. In fact, the oligonucleotides may not need to have a silencing effect at all but can just be used to deliver the gemcitabines for efficacy against disease.

It is well known that chemical modifications to the bases within an oligonucleotide siRNA structure (e.g., 2′-OMe, 2′-Fluoro, phosphorothioate modifications etc.) can stabilize the oligonucleotide against nuclease attack when administered in vivo. It is feasible that such a chemically modified oligo could be directly coupled to a targeting ligand that would allow direct delivery through binding to a receptor on a cancer cell.

Additionally, antisense oligonucleotides have been used to alter expression of select genes within a cell and these single chain oligos may also be modified by gems. These modifications (at C bases) or additions to the terminus of the sequence may be used to assist in altering the hybridization efficiency to a specific target as well as for co-delivery of gemcitabines.

Similarly, miRNAs (mimics or inhibitors) have been described that have anti-cancer effects and these could be modified by insertion of gemcitabines in their sequences for do-delivery to the same cell. Especially miRNAs that can augment the action of gemcitabine would be of interest.

Finally, aptamers are RNA or DNA oligonucleotides that can be designed and made to bind with high affinity to select targets. In some instances these can be receptors on cells, or specifically receptors that are upregulated on cancer cells. Gem nucleotides can be appended or inserted into these sequences (in place of Cs) to allow high affinity binding to a target receptor on the cancer cell surface that then allows uptake of the oligo into the cell where the gemcitabine is released to exert a therapeutic effect.

Gemcitabine modified oligonucleotides (DNA or RNA) may have utility in improving cancer treatment options in multiple cancer types.

Gemcitabine is also able to act as a radiosensitizer (improving efficacy of radiation treatment of tumors). It is therefore feasible that gemcitabine would be delivered through the modification of oligos described above to specific cancer cells. Once the gemcitabine has been delivered, the tumor is exposed to radiation which then kills the tumor cells. Sometimes this radiation exposure can further produce an immune reaction against tumor specific antigens released in the process.

So oligonucleotides containing gemcitabine or other non-nucleoside analogs can be viable treatment modalities for cancer and other diseases where non-nucleoside (or nucleotide) analogs can be effective. Another example of the use of non-nucleoside analogs or nucleotide analogs is in the treatment of viral diseases. For example, in viral diseases it may be possible to co-administer an oligonucleotide containing a non-nucleoside (or nucleotide) analog. These may include the nucleoside and nucleotide reverse transcriptase inhibitors (NRTIs) such as zidovudine, didanosine, zalcitabine, stavudine, lamivudine, abacavir, emtricitabine and tenofovir or the non-nucleoside reverse transcriptase inhibitors (NNRTIs) such as nevirapine, delavirdine, efavirenz and etravirine.

RNAi. siRNA molecules can produce additive or synergistic effects in the cells, depending on the compositions and structures of particular molecules. In certain embodiments, they produce a synergistic effect that may augment or maximize the effect, e.g., to reduce tumor growth. Double-stranded RNA has been shown to silence gene expression via RNA interference (RNAi). Short-interfering RNA (siRNA)-induced RNAi regulation shows great potential to treat a wide variety of human diseases including from cancer.

RNAi is a sequence-specific RNA degradation process that provides a relatively easy and direct way to knockdown, or silence, theoretically any gene. In naturally occurring RNA interference, a double stranded (ds) RNA is cleaved by an endonuclease into siRNA molecules, overhangs at the 3′ ends. These siRNAs are incorporated into a multicomponent-ribonuclease called RNA-induced-silencing-complex (RISC). One strand of siRNA remains associated with RISC and guides the complex towards a cognate RNA that has sequence complementary to the guider single stranded siRNA (ss-siRNA) in RISC. This siRNA-directed endonuclease digests the RNA, thereby inactivating it. Studies have revealed that the use of chemically synthesized 21-25-nucleotide (“nt” or “NT”) siRNAs exhibit RNAi effects in mammalian cells, and the thermodynamic stability of siRNA hybridization (at terminals or in the ‘middle) plays a central role in determining the molecule's function.

It is not possible at this time to predict with a high degree of confidence which of many possible candidate siRNA sequences potentially targeting a mRNA sequence of a disease gene in fact exhibit effective RNAi activity. Instead, individually specific candidate siRNA polynucleotide or oligonucleotide sequences must be generated and tested in the mammalian cell culture to determine whether the intended interference with expression of a targeted gene has occurred. The unique advantage of siRNA makes it possible to be combined with multiple siRNA duplexes to target multiple disease-causing genes in the same treatment, since all siRNA duplexes are chemically homogenous with same source of origin and same manufacturing process.

As used herein, an “siRNA molecule” is a duplex oligonucleotide, that is a short, double-stranded polynucleotide, that interferes with the expression of a gene in a cell that produces RNA, after the molecule is introduced into the cell. For example, it targets and binds to a complementary nucleotide sequence in a single stranded (“ss”) target RNA molecule, such as an mRNA, a micro RNA (miRNA) or short hairpin RNA (shRNA). The target RNA is then degraded by the cell. Such molecules are constructed by techniques known to those skilled in the art. Such techniques are described in U.S. Pat. Nos. 5,898,031, 6,107,094, 6,506,559, 7,056,704 and in European Pat. Nos. 1214945 and 1230375. By convention in the field, when an siRNA molecule is identified by a particular nucleotide sequence, the sequence refers to the sense strand of the duplex molecule.

The siRNA molecule can be made of naturally occurring ribonucleotides, i.e., those found in living cells, or one or more of its nucleotides can be chemically modified by techniques known in the art. In addition to being modified at the level of one or more of its individual nucleotides, the backbone of the oligonucleotide can be modified. Additional modifications include the use of small molecules (e.g., sugar molecules), amino acid molecules, peptides, cholesterol, and other large molecules for conjugation onto the siRNA molecule.

In one embodiment, the molecule is an oligonucleotide with a length of about 19 to about 35 base pairs. In one aspect of this embodiment, the molecule is an oligonucleotide with a length of about 19 to about 27 base pairs. In another aspect, the molecule is an oligonucleotide with a length of about 21 to about 25 base pairs. In another embodiment, the siRNA molecule or other nucleic acid has a length of 20 to 30 base pairs; in still another embodiment the siRNA molecule or other nucleic acid has a length of 24 to 28 base pairs. The molecule can have blunt ends at both ends, or sticky ends at both ends, or one of each. As discussed in more detail below, the siRNA molecules may include one or more chemical modifications at the individual nucleotide level or at the oligonucleotide backbone level, or it may have no modifications. In one advantageous embodiment a modified siRNA of the disclosed embodiments possesses strand lengths of 25 nucleotides. In another embodiment the modified siRNA possesses strand lengths of 19 to 25 nucleotides. In some embodiments, the siRNA molecules can be asymmetric where one strand is shorter than the other (typically by 2 bases, e.g., a 21mer with a 23mer or a 19mer with a 21mer or a 23mer with a 25mer). The strands may be modified by inclusion of a dTdT overhang group on the 3′ end of selected strands. In all these aspects, the molecule may have blunt ends at both ends, or sticky ends at both ends, or a blunt end at one end and a sticky end at the other.

In the composition of the disclosed embodiments, the relative amounts of the two different molecules and the copolymer can vary. In one embodiment, the ratio of the two different siRNA molecules is about 1:1 by mass. In another embodiment, the ratio of these molecules to the copolymer is about 1:4, 1:4.5, or 1:5 by mass. Advantageously, the ratio of the two different siRNA molecules is about 1:1 by mass and the ratio of these molecules to the copolymer is about 1:4, 1:4.5, or 1:5 by mass. With these ratios, the composition forms nanoparticles with an average size of about 150 nm in diameter.

In one embodiment, the siRNA molecules are selected from those identified from FIG. 1. An example is PGN7, (SEQ. ID No. 9-10), a modified siRNA-GEM RNAi agent: PGN7 sense strand: 5′-AAGAAAgAgau[GEM]uGuAu[GEM]AAu-3′(SEQ ID NO: 9); and PGN7 antisense strand: 5′-AuUGAuACAGAuCuCuUuCuU[GEM][GEM]dTdT-3′ (SEQ ID NO: 10).

The disclosed embodiments include a method for identifying the desired siRNA molecules comprising the steps of: (a) creating a collection of siRNA molecules designed to target a complementary nucleotide sequence in the target mRNA molecules, wherein the targeting strands of the siRNA molecules comprise various sequences of nucleotides; (b) selecting the siRNA molecules that show the highest desired effect against the target mRNA molecules in vitro; (c) evaluating the selected siRNA molecules in an animal wound model; and (d) selecting the siRNA molecules that show the greatest efficacy in the model for their silencing activity and therapeutic effect.

Importantly, it is presently not possible to predict with high degree of confidence which of many possible candidate siRNA sequences potentially targeting an mRNA sequence of a disease gene will, in fact, exhibit effective RNAi activity. Instead, individually specific candidate siRNA polynucleotide or oligonucleotide sequences must be generated and tested in mammalian cell culture, such as an in vitro organ culture assay, to determine whether the intended interference with expression of a targeted gene has occurred. The unique advantage of siRNA makes it possible to be combined with multiple siRNA duplexes to target multiple disease causing genes in the same treatment, since all siRNA duplexes are chemically homogenous with same source of origin and same manufacturing process.

Advantageously, the siRNA molecules are evaluated in at least two animal models. In one method embodiment, the formulation of the pharmaceutical composition comprising the RNAi agent or complex comprises the steps of adding a pharmaceutically acceptable carrier to each of the siRNA molecules to form pharmaceutical compositions and evaluating each of the pharmaceutical compositions in the animal wound model or models.

In another embodiment, the siRNA sequences are prepared in such way that each one can target and inhibit the same gene from, at least, both human and mouse, or human and non-human primate. In one aspect, the siRNA molecules bind to both a human mRNA molecule and a homologous mouse mRNA molecule. That is, the human and mouse mRNA molecules encode proteins that are substantially the same in structure or function. Other mammal animal models whose mRNA molecules encode proteins that are substantially the same in structure or function may be tested and, if viable, employed in the disclosed embodiments as well. The efficacy and toxicity reactions observed in the mouse disease models provide a good understanding about what is going to happen in humans. More importantly, the siRNA molecules tested in the mouse model are good candidates for human pharmaceutical agents. The human/mouse homology design of an siRNA drug agent can eliminate the toxicity and adverse effect of those species specificities observed in monoclonal antibody drugs.

In one embodiment, the disclosed embodiments provide a composition comprising two or more different siRNA molecules that bind to an mRNA that codes for a CHk protein in a mammalian cell. The molecules may bind to different nucleotide sequences within the target mRNA. The siRNA molecules can produce additive or synergistic effects in the cells, depending on the compositions and structures of the particular molecules. In an advantageous embodiment, they produce a synergistic effect. In certain applications of these embodiments, the siRNA molecules with GEM (e.g., SEQ. ID Nos. 1-10) or non-silencing nucleic acids comprising GEM (SEQ. ID No. 11-12) are selected from those identified in FIG. 1.

The siRNA molecules are combined with a pharmaceutically acceptable carrier to provide pharmaceutical compositions for administering to a mammal. In the disclosed embodiments, the patient or subject may be a mammal; the mammal may be a laboratory animal, such as a dog, cat, pig, non-human primate, or rodent, such as a mouse, rat, or guinea pig. In certain advantageous embodiments, the mammal is a human.

Histidine-Lysine Co-Polymers. The carrier is a histidine-lysine copolymer that forms a nanoparticle containing an siRNA molecule, wherein the nanoparticle has a size of 100-400 nm in diameter. In one aspect of this embodiment, the carrier is selected from the group consisting of the HKP species, H3K4b and PT73, which have a lysine backbone with four branches containing multiple repeats of histidine, lysine, or asparagine, for example, HKP(+H). When an HKP aqueous solution is mixed with siRNA at a N/P ratio of 4:1 by mass, the nanoparticles (average size of 100-200 nm in diameter) self-assemble. In another aspect of this embodiment, the HKP has the following formula:

(R)K(R)-K(R)-(R)K(X),
where
R = KHHHKHHHKHHHKHHHK (SEQ ID NO: 17),
or
R = KHHHKHHHNHHHNHHHN (SEQ ID NO: 18),
X = C(O)NH2, K = lysine, H = histidine,
and N = asparagine.

In still another aspect of this embodiment, the HKP has the following formula:

(R)K(R)-K(R)-(R)K(X) (SEQ ID NO: 21),
where
R = KHHHKHHHKHHHKHHHK (SEQ ID NO: 17),
X = C(O)NH2, K = lysine, H = histidine.

In still another aspect of this embodiment, the HKP has the following formula: (HKP(+H)) where the third replicating HHHK (SEQ ID NO: 19) motif has an extra H (located 6 characters from the right end)

(R)K(R)-K(R)-(R)K(X) (SEQ ID NO: 22),
where
R = KHHHKHHHKHHHHKHHHK (SEQ ID NO: 20),
X = C(O)NH2, K = lysine, H = histidine.

One particular embodiment of the disclosed embodiments provides a method of administering a pharmaceutical composition into the tissue of a human, comprising injecting directly into the tumor tissue a therapeutically effective amount of a composition comprising the siRNA molecule PGN7 with siRNA sense and antisense sequences indicated in FIG. 1 and above: PGN7 sense strand: 5′-AAGAAAgAgau[GEM]uGuAu[GEM]AAu-3′ and PGN7 antisense strand: 5′-AuUGAuACAGAuCuCuUuCuU[GEM][GEM]dTdT-3′ (together, SEQ. ID No. 9-10) and a pharmaceutically acceptable carrier comprising a pharmaceutically acceptable histidine-lysine co-polymer. In one aspect of this embodiment, the histidine-lysine co-polymer comprises the histidine-lysine co-polymer species H3K4b or the histidine-lysine co-polymer species PT73. In another aspect of this embodiment, the histidine-lysine co-polymer has the formula:

(R)K(R)-K(R)-(R)K(X) (SEQ ID NO: 21),
where
R = KHHHKHHHKHHHKHHHK (SEQ ID NO: 17),
X = C(O)NH2, K = lysine, H = histidine,
and N = asparagine.
In still another aspect of this embodiment,
the histidine-lysine co-polymer
has the formula:
(R)K(R)-K(R)-(R)K(X),
where
R = KHHHKHHHKHHHKHHHK (SEQ ID NO: 17),
or
R = KHHHKHHHKHHHHKHHHK (SEQ ID NO: 20),
X = C(O)NH2, K = lysine, H = histidine.

Another disclosed embodiment provides a method for treatment of cancer in a mammal, comprising injecting through intravenous or intratumoral routes a therapeutically effective amount of a composition comprising the modified siRNA-GEM molecule PGN6, with the following sense and antisense sequences (SEQ. ID No. 7-8) designated together, respectively, as Sense strand: 5′-AAGAAAgAgau [GEM]UGUAU[GEM]AAU-3′; and antisense strand: 5′-AuUGAuACAGAUCuCuuucuu[GEM][GEM]dTdT-3′, and a pharmaceutically acceptable carrier comprising a pharmaceutically acceptable histidine-lysine co-polymer. In one aspect of this embodiment, the histidine-lysine co-polymer comprises the histidine-lysine co-polymer species H3K4b or the histidine-lysine co-polymer species PT73. In another aspect of this embodiment, the histidine-lysine co-polymer has the formula:

(R)K(R)-K(R)-(R)K(X) (SEQ ID NO: 21),
where
R = KHHHKHHHKHHHKHHHK (SEQ ID NO: 17),
X = C(O)NH2, K = lysine, H = histidine,
and N = asparagine.

In still another aspect of this embodiment, the histidine-lysine co-polymer has the formula:

(R)K(R)-K(R)-(R)K(X), where R=KHHHKHHHKHHHKHHHK (SEQ ID NO: 17), or with an additional H located 6 characters to the right end of this sequence:

R = KHHHKHHHKHHHHKHHHK (SEQ ID NO: 20),
X = C(O)NH2, K = lysine, H = histidine.

Other HKP copolymers suitable for use in the disclosed embodiments are provided in, e.g., U.S. Pat. Nos. 7,070,807; 7,163,695; 7,465,708; and 7,772,201.

### Preparation of Formulations—Pharmaceutical Compositions

In certain embodiments, the disclosed embodiments provide for a pharmaceutical composition comprising the (double stranded, ds) RNAi agent of the disclosed embodiments. The dsRNA agent sample can be suitably formulated and introduced into the environment of the cell by any means that allows for a sufficient portion of the sample to enter the cell to induce gene silencing, if it is to occur. Many formulations for dsRNA are known in the art and can be used so long as the dsRNA gains entry to the target cells so that it can act. See, e.g., U.S. published patent application Nos. 2004/0203145 A1 and 2005/0054598 A1. For example, the dsRNA agent of the disclosed embodiments can be formulated in buffer solutions such as phosphate buffered saline solutions, liposomes, micellar structures, and capsids. Formulations of dsRNA agent with cationic lipids can be used to facilitate transfection of the dsRNA agent into cells. For example, cationic lipids, such as lipofectin (U.S. Pat. No. 5,705,188), cationic glycerol derivatives, and polycationic molecules, such as polylysine (published PCT International Application WO 97/30731), can be used. Suitable lipids include Oligofectamine, Lipofectamine (Life Technologies), NC388 (Ribozyme Pharmaceuticals, Inc., Boulder, Colo.), or FuGene 6 (Roche) all of which can be used according to the manufacturer's instructions.

Such compositions typically include the nucleic acid molecule and a pharmaceutically acceptable carrier. As used herein the language “pharmaceutically acceptable carrier” includes saline, solvents, dispersion media, coatings, antibacterial and antifungal agents, isotonic and absorption delaying agents, and the like, compatible with pharmaceutical administration. Supplementary active compounds can also be incorporated into the compositions.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be advantageous to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in a selected solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the advantageous, optional methods of preparation are vacuum drying and freeze-drying which yields a powder of the active ingredient plus any additional desired ingredient from a previously sterile-filtered solution thereof.

Oral compositions generally include an inert diluent or an edible carrier, such as HKPs, discussed infra. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using standard techniques. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

The method of introducing dsRNA agents into the environment of the cell will depend on the type of cell and the make-up of its environment. For example, when the cells are found within a liquid, one advantageous, optional formulation is with a lipid formulation such as in lipofectamine and the dsRNA agents can be added directly to the liquid environment of the cells. Lipid formulations optionally can be administered to animals by intravenous, intramuscular, or intraperitoneal injection, or orally or by inhalation or other methods as are known in the art. When the formulation is suitable for administration into animals such as mammals and more specifically humans, the formulation is also pharmaceutically acceptable. Pharmaceutically acceptable formulations for administering oligonucleotides are known and can be used. In some instances, it may be advantageous to formulate dsRNA agents in a buffer or saline solution and directly inject the formulated dsRNA agents into cells, as in studies with oocytes. The direct injection of dsRNA agent duplexes may also be done. For suitable methods of introducing dsRNA, see U.S. published patent application No. 2004/0203145 A1.

The modified siRNA molecules with GEM are mixed with a pharmaceutically acceptable carrier to provide compositions for administering to a subject. Advantageously, the subject is a human. In one embodiment, the composition comprises a pharmaceutically acceptable carrier and at least three siRNA molecules, wherein each siRNA molecule binds an mRNA molecule that encodes a gene selected from the group consisting of pro-inflammatory pathway genes, pro-angiogenesis pathway genes, and pro-cell proliferation pathway genes. In still another embodiment, each siRNA contains at least three siRNA duplexes that target at least three different gene sequences. Advantageously, each gene is selected from a different pathway. The disclosed embodiments comprise pharmaceutically effective carriers for enhancing the siRNA delivery into the disease tissues and cells.

In various embodiments of the composition, the carrier comprises one or more components selected from the group consisting of a saline solution, a sugar solution, a polymer, a lipid, a cream, a gel, and a micellar material. Further components or carriers include: a polycationic binding agent, cationic lipid, cationic micelle, cationic polypeptide, hydrophilic polymer grafted polymer, non-natural cationic polymer, cationic polyacetal, hydrophilic polymer grafted polyacetal, ligand functionalized cationic polymer, and ligand functionalized-hydrophilic polymer grafted polymer, biodegradable polyesters, such as poly(lactic acid) (PLA), poly(glycolic acid) (PGA), and poly(lactic-co-glycolic acid) (PLGA), PEG-PEI (polyethylene glycol and polyethylene imine), Poly-Spermine (Spermidine), and polyamidoamine (PAMAM) dendrimers. Optionally, the carrier is a histidine-lysine copolymer that is believed to form a nanoparticle containing an siRNA molecule, wherein the nanoparticle has a size of about 100 to 400 nm in diameter, or advantageously, 80 to 200 nm in diameter; and more advantageously, 80 to 150 nm in diameter. In some embodiments the siRNA molecule may be formulated with methylcellulose gel for topical administration. In some advantageous embodiments, the nanoparticle, of a size ranging from 80 to 150, or 80 to 200 nm and containing an siRNA molecule, may be formulated for injection or infusion without methylcellulose gel. Methods of formulating nanoparticles with a methylcellulose gel are known in the art.

The phrase “pharmaceutically acceptable carrier” or “carrier” refers to a carrier for the administration of a therapeutic agent. Exemplary carriers include saline, buffered saline, dextrose, water, glycerol, ethanol, and combinations thereof. For drugs administered orally, pharmaceutically acceptable carriers include, but are not limited to pharmaceutically acceptable excipients such as inert diluents, disintegrating agents, binding agents, lubricating agents, sweetening agents, flavoring agents, coloring agents and preservatives. Suitable inert diluents include sodium and calcium carbonate, sodium and calcium phosphate, and lactose, while corn starch and alginic acid are suitable disintegrating agents. Binding agents may include starch and gelatin, while the lubricating agent, if present, will generally be magnesium stearate, stearic acid or talc. If desired, the tablets may be coated with a material such as glyceryl monostearate or glyceryl distearate, to delay absorption in the gastrointestinal tract. The pharmaceutically acceptable carrier of the disclosed dsRNA compositions may be micellar structures, such as a liposomes, capsids, capsoids, polymeric nanocapsules, or polymeric microcapsules.

Polymeric nanocapsules or microcapsules facilitate transport and release of the encapsulated or bound dsRNA into the cell. They include polymeric and monomeric materials, especially including polybutylcyanoacrylate. A summary of materials and fabrication methods has been published (see Kreuter, 1991). The polymeric materials which are formed from monomeric and/or oligomeric precursors in the polymerization/nanoparticle generation step, are per se known from the prior art, as are the molecular weights and molecular weight distribution of the polymeric material which a person skilled in the field of manufacturing nanoparticles may suitably select in accordance with the usual skill.

Modifications and Linkages. A dsRNA agent of the disclosed embodiments can be conjugated (e.g., at its 5′ or 3′ terminus of its sense or antisense strand) or unconjugated to another moiety (e.g., a non-nucleic acid moiety such as a peptide), an organic compound (e.g., a dye, cholesterol, or the like). Modifying dsRNA agents in this way may improve cellular uptake or enhance cellular targeting activities of the resulting dsRNA agent derivative as compared to the corresponding unconjugated dsRNA agent, are useful for tracing the dsRNA agent derivative in the cell or improve the stability of the dsRNA agent derivative compared to the corresponding unconjugated dsRNA agent.

As used herein, the term “nucleic acid” refers to deoxyribonucleotides, ribonucleotides, or modified nucleotides, and polymers thereof in single- or double-stranded form. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphorodithioates, phosphoramidates, methyl phosphonates, chiral-methyl phosphonates, 2′-O-methyl ribonucleotides, 2′-Fluoro ribonucleotides, peptide-nucleic acids (PNAs) and unlocked nucleic acids (UNAs; see, e.g., Jensen et al. Nucleic Acids Symposium Series 52: 133-4), and derivatives thereof.

As used herein, “nucleotide” is used as recognized in the art to include those with natural bases (standard), and modified bases well known in the art. Such bases are generally located at the 1′ position of a nucleotide sugar moiety. Nucleotides generally comprise a base, sugar and a phosphate group. The nucleotides can be unmodified or modified at the sugar, phosphate and/or base moiety, (also referred to interchangeably as nucleotide analogs, modified nucleotides, non-natural nucleotides, non-standard nucleotides and other, see, e.g., Usman and McSwiggen, supra; Eckstein, et al., International PCT Publication No. WO 92/07065; Usman et al, International PCT Publication No. WO 93/15187; Uhlman & Peyman. There are several examples of modified nucleic acid bases known in the art as summarized by Limbach, et al, Nucleic Acids Res. 22:2183, 1994. Some of the non-limiting examples of base modifications that can be introduced into nucleic acid molecules include, hypoxanthine, purine, pyridin-4-one, pyridin-2-one, phenyl, pseudouracil, 2,4,6-trimethoxy benzene, 3-methyl uracil, dihydrouridine, naphthyl, aminophenyl, 5-alkylcytidines (e.g., 5-methylcytidine), 5-alkyluridines (e.g., ribothymidine), 5-halouridine (e.g., 5-bromouridine) or 6-azapyrimidines or 6-alkylpyrimidines (e.g. 6-methyluridine), propyne, and others (Burgin, et al., Biochemistry 35:14090, 1996; Uhlman & Peyman, supra). By “modified bases” in this aspect is meant nucleotide bases other than adenine, guanine, cytosine and uracil at 1′ position or their equivalents.

As used herein, “modified nucleotide” refers to a nucleotide that has one or more modifications to the nucleoside, the nucleobase, pentose ring, or phosphate group. For example, modified nucleotides exclude ribonucleotides containing adenosine monophosphate, guanosine monophosphate, uridine monophosphate, and cytidine monophosphate and deoxyribonucleotides containing deoxyadenosine monophosphate, deoxyguanosine monophosphate, deoxythymidine monophosphate, and deoxycytidine monophosphate. Modifications include those naturally occurring that result from modification by enzymes that modify nucleotides, such as methyltransferases. Modified nucleotides also include synthetic or non-naturally occurring nucleotides. Synthetic or non-naturally occurring modifications in nucleotides include those with 2′ modifications, e.g., 2′-methoxy, 2′-methoxyethoxy, 2′-fluoro, 2′-allyl, 2′-O-[2-(methylamino)-2-oxoethyl], 4′-thio, 4′-CH₂—O-2′-bridge, 4′-(CH₂)₂-O-2′-bridge, 2′-LNA or other bicyclic or “bridged” nucleoside analog, and 2′-O—(N-methylcarbamate) or those comprising base analogs.

In connection with 2′-modified nucleotides as described for the present disclosure, by “amino” is meant 2′-NH₂ or 2′-O—NH₂, which can be modified or unmodified. Such modified groups are described, e.g., in Eckstein et al., U.S. Pat. No. 5,672,695 and Matulic-Adamic et al., U.S. Pat. No. 6,248,878. “Modified nucleotides” of the disclosed embodiments can also include nucleotide analogs as described above.

In reference to the nucleic acid molecules of the present disclosure, modifications may exist upon these agents in patterns on one or both strands of the ds ribonucleic acid (dsRNA). As used herein, “alternating” positions refers to a pattern where every other nucleotide is a modified nucleotide or there is an unmodified nucleotide (e.g., an unmodified ribonucleotide) between every modified nucleotide over a defined length of a strand of the dsRNA (e.g., 5′-MNMNMN3′; -3′-MNMNMN-5′; where M is a modified nucleotide and N is an unmodified nucleotide). The modification pattern starts from the first nucleotide position at either the 5′ or 3′ terminus according to a position numbering convention. The pattern of modified nucleotides at alternating positions may run the full length of the strand, but in certain embodiments includes at least 4, 6, 8, 10, 12, 14 nucleotides containing at least 2, 3, 4, 5, 6 or 7 modified nucleotides, respectively. “Alternating pairs of positions” refers to a pattern where two consecutive modified nucleotides are separated by two consecutive unmodified nucleotides over a defined length of a strand of the dsRNA (e.g., 5′-MMNNMMNNMMNN-3′; 3′-MMNNMMNNMMNN-5′; where M is a modified nucleotide and N is an unmodified nucleotide). The modification pattern starts from the first nucleotide position at either the 5′ or 3′ terminus according to a position numbering convention such as those described herein. The pattern of modified nucleotides at alternating positions may run the full length of the strand, but advantageously includes at least 8, 12, 16, 20, 24, 28 nucleotides containing at least 4, 6, 8, 10, 12 or 14 modified nucleotides, respectively. It is emphasized that the above modification patterns are exemplary and are not intended as limitations on the scope of the disclosed embodiments.

As used herein, “loop” refers to a structure formed by a single strand of a nucleic acid, in which complementary regions that flank a particular ss nucleotide region hybridize in a way that the ss nucleotide region between the complementary regions is excluded from duplex formation or Watson-Crick base pairing. A loop is a ss nucleotide region of any length. Examples of loops include the unpaired nucleotides present in such structures as hairpins, stem loops, or extended loops.

In certain embodiments, the first and second oligonucleotide sequences of the siRNA or other nucleic acid exist on separate oligonucleotide strands that can be and typically are chemically synthesized. In some embodiments, both strands are 25 nucleotides in length, are completely complementary and have blunt ends. In certain embodiments of the disclosed embodiments, the modified siRNAs exist on separate RNA oligonucleotides (strands). In certain embodiments the modified siRNA agent is comprised of two oligonucleotide strands of differing lengths, with one possessing a blunt end at the 3′ terminus of a first strand (sense strand) and a 3′ overhang at the 3′ terminus of a second strand (antisense strand). The siRNA can also contain one or more deoxyribonucleic acid (DNA) base substitutions.

Suitable siRNA compositions that contain two separate oligonucleotides can be chemically linked outside their annealing region by chemical linking groups. Many suitable chemical linking groups are known in the art and can be used. Suitable groups will not block endonuclease activity on the siRNA and will not interfere with the directed destruction of the RNA transcribed from the target gene. Alternatively, the two separate oligonucleotides can be linked by a third oligonucleotide such that a hairpin structure (shRNA) is produced upon annealing of the two oligonucleotides making up the siRNA composition. The hairpin structure will not block endonuclease activity on the siRNA and will not interfere with the directed destruction of the target RNA.

The dsRNA molecules of the disclosed embodiments are added directly, or can be complexed with lipids (e.g., cationic lipids), packaged within liposomes, or otherwise delivered to target cells or tissues. The nucleic acid or nucleic acid complexes can be locally administered to relevant tissues ex vivo, or in vivo through direct dermal application, transdermal application, or injection, with or without their incorporation in biopolymers.

The dsRNA agent can be formulated as a pharmaceutical composition (formulation), which comprises a pharmacologically effective amount of a dsRNA agent and pharmaceutically acceptable carrier. The phrases “pharmacologically effective amount” and “therapeutically effective amount” or simply “effective amount” refer to that amount of an RNA effective to produce the intended pharmacological, therapeutic or preventive result. For example, if a given clinical treatment is considered effective when there is at least a 20 percent reduction in a measurable parameter associated with a disease or disorder, a therapeutically effective amount of a drug for the treatment of that disease or disorder is the amount necessary to effect at least a 20 percent reduction in that parameter.

The dosages, methods of administration, and times of administration are readily determinable by a person skilled in the art, given the teachings contained herein.

### Dosing

As defined herein, a therapeutically effective amount of a nucleic acid molecule (i.e., an effective dosage, or a therapeutically effective dosage) depends on the nucleic acid selected. For instance, single dose amounts of a dsRNA (or, e.g., a construct(s) encoding for such dsRNA) in the range of approximately 1 pg to up to 10 mg may be administered. In the disclosed embodiments, 1, 10, 30, 100, or 1000 pg, or 10, 30, 100, or 1000 ng, or 10, 30, 100, or 1000 μg, may be administered in one or more areas of the body of a 60 to 120 kg subject.

More particularly, dosages administered according to the disclosed embodiments are between about 5 and about 170 μg, between about 10 and about 160 μg, between about 10 and about 130 μg, between about 10 and about 70 μg, between about 10 and about 40 μg, between about 20 and about 50 μg, between about 20 and about 30 μg, between about 30 and about 70 μg, between about 40 and about 80 μg, between about 60 and about 90 μg, between about 50 and about 100 μg, between about 70 and about 100 μg, and between about 80 and about 120 μg, at least once weekly for one to 12 weeks.

Dosages and treatment periods will vary. In some embodiments, doses ranging from 60 to 150 μg are administered. Advantageously, the compositions are administered subcutaneously or subdermally in multiple areas where remodeling of the adipose tissue is desired, for example, in submental or adipose tissue. Each dose may be, for example, 60-150 μg per cm² administered to several areas in a 60 kg to a 120 kg patient. The skilled artisan will recognize that doses greater or lesser than 60-150 μg per cm² may be administered, for example, between 10-300 μg per cm².

The compositions can be administered from one or more times per day to one or more times per week for the desired length of the treatment from one week up to several months or a year or more; dosages may be administered in some embodiment once every other day. The skilled artisan will appreciate that certain factors may influence the dosage and timing required to effectively treat a subject, including but not limited to the severity of the disease or disorder, previous treatments, the general health and/or age of the subject, and other diseases present. Treatment of a subject with a therapeutically effective amount of a nucleic acid (e.g., dsRNA), protein, polypeptide, or antibody can include a single treatment or, advantageously, can include a series of treatments. In an advantageous embodiment, one or more doses is administered weekly or semiweekly for a period between one and six to 12 weeks. In another embodiment, one or more doses is administered daily.

In general, based on a per kg body weight unit, a suitable dosage of dsRNA may range between 1 ng to 2 milligrams per kilogram body weight of the recipient per day, week or month, but more likely will be within the narrower range of between about 0.01 to about 20 micrograms per kilogram body weight per day, week or month, or in the range between about 0.001 to about 5 micrograms per kilogram of body weight per day, week or month, or in the range between about 1 to about 500 nanograms per kilogram of body weight per day, week or month, or in the range between about 0.01 to about 10 micrograms per kilogram body weight per day, week or month, or in the range between about 0.10 to about 5 micrograms per kilogram body weight per day, week or month, or in the range between about 0.1 to about 2.5 micrograms per kilogram body weight per day, week or month. A pharmaceutical composition comprising the dsRNA can be administered once daily. However, the therapeutic agent may also be dosed in units containing two, three, four, five, six or more sub-doses administered at appropriate intervals throughout the day, week or month. In that case, the dsRNA contained in each sub-dose must be correspondingly smaller to achieve the total daily, weekly or monthly dosage unit. The dosage unit can also be compounded for a single dose over several days, e.g., using a conventional sustained release formulation which provides sustained and consistent release of the dsRNA over a several day period. Sustained release formulations are well known in the art. In this embodiment, the dosage unit contains a corresponding multiple of the daily dose. Regardless of the formulation, the pharmaceutical composition must contain dsRNA in a quantity sufficient to inhibit expression of the target gene in the animal or human being treated. The composition can be compounded in such a way that the sum of the multiple units of dsRNA together contain a sufficient dose.

Depending on the particular target gene sequence and the dose of dsRNA agent material delivered, this process may provide partial or complete loss of function for the target gene. A reduction or loss of expression (either target gene expression or encoded polypeptide expression) in at least 50%, 60%, 70%, 80%, 90%, 95% or 99% or more of targeted cells is exemplary. Inhibition of target gene levels or expression refers to the absence (or observable decrease) in the level of target gene or target gene-encoded protein. Specificity refers to the ability to inhibit the target gene without manifest effects on other genes of the cell. The consequences of inhibition can be confirmed by examination of the outward properties of the cell or organism or by biochemical techniques such as RNA solution hybridization, nuclease protection, Northern hybridization, reverse transcription, gene expression monitoring with a microarray, antibody binding, enzyme linked immunosorbent assay (ELISA), Western blotting, radioimmunoassay (RIA), other immunoassays, and fluorescence activated cell analysis (FACS). Inhibition of target target gene sequence(s) by the dsRNA agents of the disclosed embodiments also can be measured based upon the effect of administration of such dsRNA agents upon development/progression of a target gene associated disease or disorder, e.g., deleterious adipose tissue remodeling due to obesity, over feeding or a metabolic derangement, tumor formation, growth, metastasis, etc., either in vivo or in vitro. Treatment with pharmaceutical compositions comprising modified siRNAs with GEM can result in significant reductions in tumor volume or in cancer cell levels of, e.g., up to 10%, 20%, 30%, 40%, 50%, 60%, 70%, 80%, 90%, 95% or 99% or more. Improvements can also be measured in logarithmic terms, e.g., 10-fold, 100-fold, 1000-fold, 10⁵-fold, 10⁶-fold, 10⁷-fold reduction in cancer cell levels could be achieved via administration of the modified dsRNA molecules (with GEM) of the disclosed embodiments to cells, a tissue, or a subject. For example, in some embodiments a pharmaceutical composition (formulation) may be administered intratumorally, subcutaneously or systemically through an intravenous injection to effect at least a 20, 25, 30, 35, 40, 45, 50, 55, 60, 65, 70, 75, 80, 85, 90, 95, 96, 97, 98, 99 or 100 percent reduction in the volume of the tumor(s). In some embodiments, treatment of a subject with a pharmaceutical composition comprising one or more modified siRNAs with GEM result in a reduction in the advancement of the cancer by at least 20 percent, 30 percent, 40 percent, 50 percent, 60 percent 70 percent, 80 percent, 90 percent, 95 percent or 100 percent.

The data obtained from the cell culture assays and animal studies (toxicity, therapeutic efficacy) can be used in formulating a range of dosages for use in humans and other mammals. Each dosage of such compounds lies advantageously within a range of circulating concentrations that include the ED₅₀ with little or no toxicity. The dosage may vary within this range depending upon the dosage form employed and the route of administration utilized. For a compound used in the method of the disclosed embodiments, the therapeutically effective amount can be estimated initially from cell culture assays. A dose may be formulated in animal models to achieve a circulating plasma concentration range that includes the IC₅₀ (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

### Administration

Suitably formulated compositions of the disclosed embodiments can be administered by means known in the art such as by parenteral routes, including intravenous, intramuscular, intraperitoneal, subcutaneous, subdermal, transdermal, airway (aerosol), rectal, vaginal and topical (including buccal and sublingual) administration. In some embodiments, the pharmaceutical compositions are administered by intravenous or intra-parenteral infusion or injection. In one embodiment, the composition is administered by injection into the tissue, e.g., intratumoral. In another embodiment, the composition is ministered by subcutaneous injection into a mammal. In still another embodiment, the composition is administered topically to the mammal. In certain advantageous embodiments, intravenous administration is an optional route. In other embodiments, intratumoral administration may be more effective. A therapeutically effective amount of the pharmaceutical composition is delivered to the tissue, which may include, but is not limited to, skin, liver, lung, kidney, and heart tissue.

A formulation is prepared to be compatible with its intended route of administration. Examples of routes of administration include parenteral, e.g., intravenous, intradermal, subcutaneous, subdermal, oral (e.g., inhalation), transdermal (topical), transmucosal, intratumoral and rectal administration. Solutions or suspensions used for parenteral, intratumoral, intradermal, subdermal or subcutaneous application can include the following components: a sterile diluent such as water for injection, saline solution, fixed oils, polyethylene glycols, glycerin, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The siRNA formulations can also be administered by transfection or infection using methods known in the art, including but not limited to the methods described in McCaffrey et al. (2002), Nature, 418(6893), 38-9 (hydrodynamic transfection); Xia et al. (2002), Nature Biotechnol., 20(10), 1006-10 (viral-mediated delivery); or Putnam (1996), Am. J. Health Syst. Pharm. 53(2), 151-160, erratum at Am. J. Health Syst. Pharm. 53(3), 325 (1996).

Further, the siRNA formulations can also be administered by a method suitable for administration of nucleic acid agents, such as a DNA vaccine. These methods include gene guns, bio injectors, and skin patches as well as needle-free methods such as the micro-particle DNA vaccine technology disclosed in U.S. Pat. No. 6,194,389, and the mammalian transdermal needle-free vaccination with powder-form vaccine as disclosed in U.S. Pat. No. 6,168,587. Additionally, intranasal delivery is possible, as described in, inter alia, Hamajima et al. (1998), Clin. Immunol. Immunopathol., 88(2), 205-10. Liposomes (e.g., as described in U.S. Pat. No. 6,472,375) and microencapsulation can also be used. DODAB may be used to improve uptake. Biodegradable targetable microparticle delivery systems can also be used (e.g., as described in U.S. Pat. No. 6,471,996).

### Treating Cancers

Typically, targeted deliver of siRNA focuses on silencing a single gene. Cancers, however, are polygenic and as a result advance through the effects of multiple genes. Unfortunately, tumors can continue to grow in the face of silencing a single targeted gene when they become resistant to the inhibition of a particular gene due to selection of tumor cells that do not rely on that gene for advancement. As result, either a very potent single siRNA molecule must be used or, advantageously, one may leverage the synergy produced when at least two siRNA molecules are made available in small molecules such as nanoparticles.

The modified siRNA molecules with GEM disclosed herein may be used to treat any number of types of cancer, e.g., pancreatic, lung, colon, breast, gastric, ovarian and urinary bladder cancer. In one embodiment combinations of modified siRNA with GEM in combination with at least one additional siRNA, with or without modifications and with or without GEM may be used for an additive or synergistic effect to treat cancer. Administration in some embodiments will be intravenous, while in others administration will be intratumoral. In still others, the route of administration will vary depending on the type of cancer, among other factors. In certain embodiments, PGN6 or PGN7 in combination with BCLxL or Wee1 or other siRNAs may be successful in attenuating the growth of tumors, preventing their growth entirely, or even reducing volume of existing tumors. In other embodiments tumor volume as well as expression levels of one or more targeted genes may be reduced between 20 and 100 percent. In still other embodiments, separate treatments for two or more types of cancer may be approached using individual treatments, each comprising a single or a combination of modified siRNAs with GEM and histidine-lysine copolymer carriers to reduce tumor volume resulting from two or more types of cancer. Any number of embodiments comprising variations on the modifications, the siRNA sequences with GEM, the histidine-lysine copolymer structures, and other additives, routes of administration, dosages and treatment schedules as disclosed may be used to treat any of a variety of cancerous tumors under this disclosure.

The presently disclosed embodiments provide for pharmaceutical compositions as well as therapeutic methods of treating a subject with cancer. Disclosed embodiments could even address prophylactic administration of the pharmaceutical compositions disclosed to those subjects at risk for developing a cancer in which tumors are present where the compositions can reduce the expression of select genes that drive tumor growth but that are not critical for normal cells.

“Treatment”, or “treating” as used herein, is defined as the application or administration of a therapeutic agent (e.g., a dsRNA agent or vector or transgene encoding same) to a patient, or application or administration of a therapeutic agent to an isolated tissue or cell line from a patient, who has the disease or disorder, a symptom of disease or disorder or a predisposition toward a disease or disorder, with the purpose to cure, heal, alleviate, relieve, alter, remedy, ameliorate, improve or affect the disease or disorder, the symptoms of the disease or disorder, or the predisposition toward disease.

In one aspect, the disclosed embodiments provide a method for preventing in a subject, a disease or disorder as described above (including, e.g., prevention of the commencement of transforming events within a subject via inhibition of TGFβ1 and Cox-2 expression), by administering to the subject a therapeutic agent (e.g., a dsRNA agent or vector or transgene encoding same). Subjects at risk for the disease can be identified by, for example, one or a combination of diagnostic or prognostic assays as described herein. Administration of a prophylactic agent can occur prior to the detection of, e.g., cancer in a subject, or the manifestation of symptoms characteristic of the disease or disorder, such that the disease or disorder is prevented or, alternatively, delayed in its progression.

The dsRNA molecules (siRNA) can be used in combination with other treatments to treat, inhibit, reduce, or prevent a deleterious adipose remodeling in a subject or organism.

Another aspect of the disclosed embodiments pertains to methods of treating subjects therapeutically, i.e., altering the onset of symptoms of the disease or disorder. These methods can be performed in vitro (e.g., by culturing the cell with the dsRNA agent) or, alternatively, in vivo (e.g., by administering the dsRNA agent to a subject).

With regards to both prophylactic and therapeutic methods of treatment, such treatments may be specifically tailored or modified, based on knowledge obtained from the field of pharmacogenomics. “Pharmacogenomics”, as used herein, refers to the application of genomics technologies such as gene sequencing, statistical genetics, and gene expression analysis to drugs in clinical development and on the market. More specifically, the term refers the study of how a patient's genes determine his or her response to a drug (e.g., a patient's “drug response phenotype”, or “drug response genotype”). Thus, another aspect of the disclosed embodiments provides methods for tailoring an individual's prophylactic or therapeutic treatment with either the target TGFβ1 and Cox-2 genes or modulators according to that individual's drug response genotype. Pharmacogenomics allows a clinician or physician to target prophylactic or therapeutic treatments to patients who will most benefit from the treatment and to avoid treatment of patients who will experience toxic drug-related side effects.

Therapeutic agents can be tested in a selected animal model. For example, a dsRNA agent (or expression vector or transgene encoding same) as described herein can be used in an animal model to determine the efficacy, toxicity, or side effects of treatment with said agent. Alternatively, an agent (e.g., a therapeutic agent) can be used in an animal model to determine the mechanism of action of such an agent.

The disclosed embodiments described and claimed are not to be limited in scope by the specific advantageous embodiments referenced herein, since these embodiments are intended as illustrations, not limitations. Any equivalent embodiments are intended to be within the scope of this disclosure, and the embodiments disclosed are not mutually exclusive. Indeed, various modifications to the embodiments, in addition to those shown and described herein will become apparent to those skilled in the art from the foregoing description. Such modifications are also intended to fall within the scope of the appended claims.

The terms and words used in the following description and claims are not limited to conventional definitions but, rather, are used to enable a clear and consistent understanding of the disclosure. Accordingly, it should be apparent to those skilled in the art that the description of various embodiments is provided for illustration purpose only and not for the purpose of limiting the disclosure with respect to the appended claims and their equivalents.

It is to be understood that the singular forms “a,” “an,” and “the” include the plural forms unless the context clearly dictates otherwise, e.g., reference to “a dermatologically active compound” includes reference to one or more such compounds.

Unless otherwise defined herein, all terms used have the same meaning as commonly understood by a person of ordinary skill in the art. Terms used herein should be interpreted as having meanings consistent with their meanings in the context of the relevant art.

As used herein, the terms “comprising,” “comprise” or “comprised,” in reference to defined or described elements of any item, composition, formulation, apparatus, method, process, system, etc., are intended to be inclusive or open ended, and includes those specified elements or their equivalents. Other elements can be included and still fall within the scope or definition of the defined item, composition, etc.

The term “about” or “approximately” means within an acceptable error range for the particular value as viewed by one of ordinary skill in the art; this depends in part on how the value is measured or determined based on the limitations of the measurement system.

“Co-administer” or “co-deliver” refers to the simultaneous administration of two pharmaceutical formulations in the blood or other fluid of an individual using the same or different modes of administration. Pharmaceutical formulations can be concurrently or sequentially administered in the same pharmaceutical carrier or in different ones.

The terms “subject,” “patient,” and “individual” are used interchangeably.

The following examples illustrate certain aspects of the disclosed embodiments and should not be construed as limiting the scope thereof.

### EXAMPLES

### Example 1: In Vitro Study Evaluating the Effect of a Variety of prChk1-PolyGEM Variants on IC₅₀

MIA PaCa-2, MDA-MB 31, LS180, HT1376, HT29, H358, H1299, OVCAR-3, HTB-9 and Panc 10.05 cells were obtained from ATCC (Rockville, MD). MiaPaCa cells were cultured in DMEM medium supplemented with 10% FBS. H358, H1299, HTB-9, N87 and Panc 10.05 cells were cultured in DMEM/10% FBS; LS180 and HT1376 in EMEM/10% FBS; HT29 cells in McCoy5A/10% FBS; OVCAR-3 cells were incubated in RPMI/20% FBS. All cells were maintained at 37 degrees C. in an atmosphere containing 5% CO².

The day before transfection, cells were seeded in a 384-well plate at an optimal density of between 300 and 800 cells/well. The next day, ten 3-fold serial dilutions of single, modified-backbone siRNAs or PGN6/PGN7 siRNA and Gemcitabine (GEM) mixed with siRNAs: Wee1 #7, KRAS #1, MCL1 #3 or DM2-P47 siRNAs at 1:1 ratio were prepared and combined with diluted Lipofectamine RNAiMAX (Life Technologies, Carlsbad, CA). Within 20 minutes, 10 μl of the transfection mixtures were added to the cells in a 40 μL medium. The final concentrations of siRNAs or Gemcitabine in the wells ranged from 100 nM to 0.002 nM.

Ninety-six hours following the addition of GEM or transfection mixtures, the number of viable cells was determined with CellTiter-Glo®-2.0 reagent (Promega, Madison, WI) by measuring luminescent signal using a Cytation 5 plate reader (BioTek Inc, Winooski, VT). The treatment group (n=4) mean (±SD) was compared to those of non-treated controls and were reported as relative cell viability.

FIG. 1 (a) shows the sequences of several prChk1-GEM variants and non-silencing siRNA+GEMs. FIG. 1 (b) shows a few additional sequences for siRNAs used in these examples. FIG. 2 shows the effect of polyGEM variants on MiaPaCa-2 cell viability, demonstrating an increasingly significant effect on IC₅₀ from by PNG6 (0.0.4 nM), STD1 (0.02 nM), and PNG7 (0.006 nM) versus Gemcitibine (8 nM), each in combination with lipofectamine.

### Example 2: Effect of PGN6 with and without Lipofectamine on Cell Viability in Pancreatic, Lung and Colon Cancer Cell Lines

Using the same experimental conditions as are described above in Example 1, the efficacy on cell viability was evaluated for PGN6 (SEQ. ID Nos. 7-8), with and without lipofentamine (“lipo”) in three types of cancer cell lines. FIG. 3 (a)-(c) show the effects of PGN6, with and without lipo on cell viability at increasing siRNA levels in pancreatic cancer cell lines. FIG. 4 (a)-(b) similarly show the effects of these variants on cell viability in lung cancer cell lines; FIG. 5 (a)-(b) show the effects of these variants on cell viability in colon cancer cell lines. All data are shown as mean±SD. Both variants show potency and efficacy against pancreatic, lung and colon cancer cells. In the absence of lipofectamine, the PNG6 products produce their effects as a result of release of the gemcitabine (GEM) from the structure. The change in IC₅₀ in the absence of lipofectamine is typically 10- to 1000-fold.

### Example 3: Effect of STD1, PGN6 and PGN7 Variants on Cell Viability in Pancreatic, Lung and Colon Cancer Cell Lines

Using the same experimental conditions as are described above in Example 1, the efficacy of STD1 (SEQ. ID Nos. 1-2), PGN6 (SEQ. ID Nos. 7-8) and PGN7 (SEQ. ID Nos. 9-10) were evaluated in three types of cancer cell lines. FIG. 6 (a)-(e) show the efficacy of STD1, PGN6 and PGN7 siRNAs in pancreatic lung and colon cancer cell lines, compared to non-silencing siRNA used as a control. Data are shown as mean±SD.

### Example 4: Effect of PGN7, WEE1 and the Combination of PGN7 and WEE1 Variants on Cell Viability in Pancreatic, Lung and Colon Cancer Cell Lines

Using the same experimental conditions as are described above in Example 1, the efficacy of PGN7 (SEQ. ID No. 9-10) alone and its combination with WEE1 #7 (SEQ. ID No. 13) was evaluated in three types of cancer cell lines. FIG. 7 (a)-(e) show the efficacy of PGN7 and the combination of PGN7 and WEE1 #7 siRNAs in pancreatic, lung, and colon cancer cell lines. Data are shown as mean±SD.

### Example 5: Effect of PGN7, BCLxL and the Combination of PGN7 and BCLxL Variants on Cell Viability in Pancreatic, Lung and Colon Cancer Cell Lines

Using the same experimental conditions as are described above in Example 1, the efficacy of PGN7 (SEQ. ID No. 9-10) and the combination of BCLxL #5 (SEQ. ID No. 14) and PGN7 was evaluated in three types of cancer cell lines. FIG. 8 (a)-(e) show the efficacy of the PGN7 variant and its combination with BCLxL in pancreatic and lung, but not colon, cancer cell lines. Data are shown as mean±SD.

### Example 6: Effect of PGN7, BCLxL and the PBH7+BCLxL Combination Variants on Cell Viability in Urinary Bladder, Gastric and Ovarian Cancer Cell Lines

Using the same or similar experimental condition as are described above in Example 1, the significant efficacy PGN7 (SEQ. ID No. 9-10)+NS (i.e., with non-silencing siRNA), BCLxL #5 (SEQ. ID No. 14)+NS, the PGN7+BCLxL combination are shown in HT1376 (400 cells/well) and HTB-9 (800 cells/well) urinary bladder cancer cell lines, N87 (600 cells/well) and OVCAR-3 (800 cells/well) in FIG. 9 (a)-(d). Data are shown as mean±SD.

### Example 7: Effect of PGN7, Wee1 or their Combination on Cell Viability in Several Urinary Bladder, Gastric and Ovarian Cancer Cell Lines

Using the same or similar experimental condition as are described above in Example 1, the significant efficacy of PGN7 (SEQ. ID No. 9-10), Wee1 #7 (SEQ. ID No. 13) or their combination are shown in HT1376 (400 cells/well) and HTB-9 (800 cells/well) urinary bladder cancer cell lines, N87 (600 cells/well) gastric cancer cell line and OVCAR-3 (800 cells/well) ovarian cancer line in FIG. 10 (a)-(d). Data are shown as mean±SD.

### Example 8: Effect of PGN7, Wee1, BCLxL and their Combinations on Cell Viability in Urinary Bladder, Gastric and Ovarian Cancer Cell Lines

Using the same or similar experimental condition as are described above in Example 1, the effects of PGN7 (SEQ. ID No. 9-10), Wee1 (SEQ. ID No. 13), BCLxL #5 (SEQ. ID No. 14) or the PGN7+Wee1, or PGN+BCLxL combinations were evaluated and are shown in HT1376 (400 cells/well) and HTB-9 (800 cells/well) urinary bladder cancer cell lines, N87 (600 cells/well) gastric cancer cell line, and OVCAR-3 (800 cells/well) ovarian cancer line in FIGS. 11 (a)-(d). Data are shown as mean±SD.

### Example 9: An In Vivo Animal Study to Evaluate the Therapeutic Efficacy of PGN6+DODAB siRNA on Pancreatic Tumor Growth in a Nonobese Diabetic-Severe Combined Immunodeficiency Mouse Model (NOD/SCID) Inoculated with a Human Pancreatic Cancer Cell Line, MIA PaCa-2

Twenty, 6- to 9-week old NOD/SCID female mice were purchased from the Jackson Laboratory. Each mouse was tagged for identification at the beginning of the study. Up to 5 mice were housed in a single cage with standard corn cob bedding for a minimum of three days for stabilization; bedding was changed every two weeks. Fluorescent lighting was provided on a 12-hour cycle; temperature was maintained between 68-79 degrees F. at 30-70 percent humidity. Mice were fed an 18 percent soy irradiated rodent feed (Envigo) and autoclaved acidified water (pH 2.5-3) were available to all animals ad libitum. The mice were observed daily for clinical signs of disease.

Tumor cell preparation: Cryogenic vials containing tumor cells were thawed and cultured according to the manufacturer's protocol. On the day of inoculation, cells were washed in a serum-free media, counted, and resuspended in cold serum-free media at a concentration of 3 million cells per 100 microliters. The Matrigel-cell mixture was kept on ice during transport to the vivarium. Cells were prepared for injection by withdrawing the Matrigel-cell mixture into a chilled 1 mL slip-tip syringes, which were kept on ice to avoid solidification of the Matrigel.

Tumor implantation: Animals were shaved and ear tagged prior to inoculation. Each mouse was immobilized and the site of injection was disinfected using an alcohol swab after which 100 mL of the cell suspension was subcutaneously injected into the rear flank of the mouse. The mice were injected with the cell suspension and left undisturbed for seven days before observing them for tumor growth.

Tumor measurement: Mice were monitored weekly for palpable tumors or changes in appearance or behavior. Once tumors were palpable they were measured weekly using calipers. Tumor volume was calculated using the equation:

$$( longest diameter \times shortest {diameter}^{2} ) / 2 = tumor volume$$

Once tumors were on average between 150-250 mm³ in volume, tumors and body weights were measured at least twice weekly for the duration of the study. One person was responsible for measuring all mice during the study.

Test article storage: Both the test article (PGN6 (SEQ. ID Nos. 7-8)+DODAB siRNA in HKP(+H) copolymer carrier) provided to the treatment group and the vehicle (DODAB siRNA+HKP (+H) copolymer carrier) provided to the control group were stored at −20 degrees C. and were light sensitive. Post-thaw storage temperature and post-formulation storage temperature was 4 degrees C. and the compositions were light sensitive. Each injection is provided from a new vial. The test article and dosing solution remaining after the study were saved for future studies.

Randomization and dose selection: Once the average tumor volume reached 150-250 mm³, mice were randomized to control (1) and treatment (2) groups and inoculated intravenously with three million cells suspended in 0.1 mL of 1:1 PBS: Matrigel. All mice were inoculated within 24 hours of randomization. The treatment group (2) was dosed with PGN6+DODAB siRNA with an HKP(+H) copolymer carrier through the IV route twice weekly for three weeks at a dose volume of 2 mg/kg (dose vol: 4 mL/kg), the control group received vehicle (DODAB siRNA+HKP(+H) copolymer carrier) IV at the same frequency, duration and volume. Day 0 was defined as the beginning of dosing. The study was terminated four weeks after administration of the first dose.

Body weight measurement and guidelines: All mice were weighed, and tumor volume was measured at least twice weekly following randomization and initiation of treatment. Body weight loss greater than 20 percent from baseline was established as the trigger for daily monitoring for signs of recovery for up to 72 hours; dosing holidays or nutritional supplements were provided based on an assessment of animal health; if no signs of recovery were observed the animal was sacrificed for humane reasons per the IACUC protocol regulations.

Clinical observations: Clinical observations were performed twice weekly at the time of tumor and body weight measurements and detailed in the study record. The mice were checked for adverse effects of tumor growth and treatments on behavior, such as mobility, food and water consumption, eye/hair matting or other abnormalities. Any mouse exhibiting signs of pain or distress was euthanized.

Moribundity: Mice exhibiting tumor sizes greater than 2000 mm³ were euthanized, as were any animals exhibiting signs of pain or distress, or animals that had exhibited more than 20 percent of body weight without signs of recovery within 72 hours.

Final Disposition: At study termination, all living animals were euthanized.

### Results

FIG. 12 (a): shows the mean (±SE) pancreatic tumor volume in NOD/SCID mice over the three week treatment period in control (line showing rising tumor growth over time) and treatment (line with little or no change over time) groups showing a significant effect of PGN6 in HKP(+H) copolymer carrier to attenuate or prevent tumor growth in the treated animals.

FIG. 12 (b) shows the mean (±SD) body weight of the NOD/SCID mice inoculated with human pancreatic cancer cells. Once tumors were palpable, treatment (2 mg/kg twice weekly) began at Day 0 in treatment (PGN6+non-silencing DODAB siRNA+HKP(+H) carrier) or control (non-silencing DODAB siRNA+HKP(+H) carrier) groups (n=10). The fact that mean body weights remained unchanged for both of the control and treatment groups over the treatment period (20 days) while the control group exhibited a significant increase in tumor volume (4×) over the same period suggests that either tumor weight did not contribute significantly to the body weight of the mice, or if it had, that the increased size/weight of the tumors in the control group may have compensated for body weight loss in that group. In contrast, in the PGN6-treated group, body weight remained constant and the tumors did not grow.

### Example 10: Effect of PGN7 in Combination with BCLxL on Cell Viability in Triple Negative Breast Cancer (MDA-MB231) and Ovarian Cancer (OVCAR3) Cell Lines

Using the same or similar experimental condition as are described above in Example 1, the effects of (i) PGN7 (SEQ. ID No. 9-10) plus a non-signaling oligonucleotide (“NS”), (ii) BCLxL #5 (SEQ. ID No. 14)+NS, and (iii) PGN7 and BCLxL #5 in combination were evaluated and are shown in triple negative breast cancer (MDA-MB231) and ovarian cancer (OVCAR3) cell lines in FIGS. 13 (a)-(b). Four hundred cells per well were used in MDA-MB231 cell lines and 800 cells per well in OVCAR3 cell lines. Data are shown as mean±SD.

As shown in both FIG. 13(a, MDA-MB231) and (b, OVCAR3) a pronounced, synergistic effect of PGN7+BCLxL #5 (IC₅₀ in (a): 0.06 nM; in (b): 0.1 nM) can be seen in both cell line types, compared to either PGN7 (IC₅₀ in (a): 1.1 nM; in (b): 0.5 nM) or BCLxL #5 (IC₅₀ in (a):0.5 nM; in (b) 2.7).

### Example 11: Effects of STD1 and PGN7 on Xenograft Tumor Growth in NU/NU Mice after Subcutaneous Implantation with MiaPaCa Tumors Cells

Twenty-four NU/NU nude mice were purchased from Charles River Laboratories (Frederick, MD) and each housed in a single cage with standard bedding, lighting and ad libitum feed.

The human pancreatic cell carcinoma MiaPaCa-2 cell line was obtained from American Type Culture Collection (ATCC, CRL-1420™) and cultured in DMEM with 10 percent fetal bovine serum and 1 percent pen-strep in a humidified incubator with temperature at 37 degrees C., and 5 percent CO₂. Subcultures were made when cell volume reached ˜80-90 percent (roughly 3-5 days). Media was changed every 3 to 4 days. All experiments were performed when the cells reached approximately 80-90 percent confluence.

MiaPaCa tumor cells (2.5×106 cells) in 100 μl of growth media and 100 μl of Matrigel were injected subcutaneously into the left flank of each mouse on Day 0. Body weight measured using a digital scale, and tumor volume measured using a digital caliper, were recorded daily. Tumor volume was measured using external digital calipers with modified ellipsoid formula: ½(length×width²).

The mice were assigned to one of three groups (n=8) approximately five to ten days after subcutaneous implantation of MiaPaCa cells, when tumors had reached 100 mm³ in volume. Twice weekly (Mon. and Thurs.) for three-four weeks, mice in all three groups received intratumoral injections of 80 μl each. Mice in the control group were injected with non-silencing (NS) siRNA with HKP(+H) as a carrier. Mice in one of the treatment groups were injected with PGN7, i.e., GEM siRNA with HKP(+H)shk1 (2.z), (SEQ. ID No. 9-10). Mice in the second treatment group were injected with STD1, i.e., GEM siRNA with HKP(+H) chk1 (2.z) (SEQ. ID No. 3-4). All injections were given under isoflurane anesthesia to minimize discomfort

Tumor volume (mm³) and body weights were measured on Days 0, 4, 7, 11, 14, 18, 21 and 25. Mean±SE for each group (PGN7, STD1 and control) were statistically compared using analysis of variance (ANOVA) with the Tukey test. Mice were also observed for excess growth, adverse reactions to treatments and other observable, adverse signs of health. Treatments were blinded to the tracers and data analysts.

All mice were sacrificed and the implanted xenograft removed on Day 26 after initial treatment (the last treatment given on Day 25). One-third of the xenograft was fixed in 0.9 percent formalin for histological analysis; one-third was frozen and one-third was preserved in RNAlater stabilization and storage solution.

As shown in FIG. 14 (a) tumor weights (mean±SE) did not differ between STD1, PGN7 and control groups (n=8) (p>0.05). Similarly, as shown in FIG. 14 (b), while mean (±SE) body weights of the two treatment groups and one control group did not differ over 25 days (p>0.05). PGN7 was effectively attenuated the increase in mean (±SE) tumor volume as compared to the growth in the control group (p<0.02) over three and a half weeks (FIG. 14 (c)).

We claim:

1. A pharmaceutical composition comprising a modified, interfering ribonucleic acid (RNAi) molecule combined with one or more gemcitabine molecules in the place of cytidine molecules, and further comprises a histidine-lysine copolymer carrier, wherein the RNAi is selected from the group consisting of siRNA, miRNA, and shRNA and mRNA.

2. The pharmaceutical composition according to claim 1, wherein the histidine-lysine copolymer carrier comprises HKP or HKP(+H).

3. The pharmaceutical composition according to claim 1, wherein the modified RNAi comprises a modification selected from the group consisting of a 2′-OMe modification, a 2′-Fluoro modification and a phosphorothioate modification.

4. The pharmaceutical composition according to claim 1, wherein the RNAi molecule comprises a combination of sequences selected from the group consisting of SEQ ID Nos. 1-2, SEQ ID Nos. 3-4, SEQ ID Nos. 5-6, SEQ ID Nos. 7-8, and SEQ ID Nos. 9-10, and wherein the composition optionally comprises lipofectamine.

5. The pharmaceutical composition according to claim 4, wherein the pharmaceutical composition further comprises at least one additional RNAi molecule comprising SEQ ID No. 13 or SEQ ID No. 14.

6. The pharmaceutical composition according to claim 1, wherein the RNAi molecule is a modified, double stranded siRNA comprising gemcitabine, wherein the siRNA comprises a combination of sequences selected from the group consisting of SEQ ID Nos. 1-2, SEQ ID Nos. 3-4, SEQ ID Nos. 5-6, SEQ ID Nos. 7-8, and SEQ ID Nos. 9-10, and wherein the composition further comprises an RNAi molecule comprising SEQ ID No. 13 or SEQ ID No. 14.

7. A method of treating a subject having cancer, comprising:
administering to the subject a therapeutically effective amount of a pharmaceutical composition comprising a modified siRNA molecule comprising gemcitabine;
wherein the composition further comprises a histidine-lysine copolymer carrier; and
wherein the siRNA molecule further comprises at least one sequence selected from the group consisting of SEQ ID Nos. 1-2, SEQ ID Nos. 3-4, SEQ ID Nos. 5-6, SEQ ID Nos. 7-8, SEQ ID Nos. 9-10, SEQ ID No. 13, and SEQ ID No. 14,
wherein the subject is treated by said administration of the pharmaceutical composition, resulting in attenuated or inhibited growth of said cancer.

8. The method of treatment according to claim 7, wherein the cancer is selected from the group consisting of breast, colon, gastric, pancreatic, lung, ovarian and urinary bladder cancer.

9. The method of treatment according to claim 7, wherein administration of an effective amount of the pharmaceutical composition comprises a method selected from the group consisting of intravenous, intratumoral and subcutaneous injection.

10. The method of treatment according to any of claim 7, wherein said pharmaceutical composition comprises lipofectamine.

11. A method of treating a subject having cancer comprising: administering to the subject the pharmaceutical composition according to claim 1.

12. The method according to claim 11, wherein the cancer is selected from the group consisting of breast, colon, gastric, pancreatic, lung, ovarian and urinary bladder cancer.

13. A method of treating a subject having cancer comprising administering to the subject a therapeutically effective amount of a pharmaceutical composition comprising at least one RNAi molecule selected from the group consisting of SEQ ID Nos. 7-8, SEQ ID Nos. 9-10, SEQ ID No. 13 and SEQ. ID No. 14, wherein the composition further comprises HKP(+H) and wherein the pharmaceutical composition is administered through intravenous or intratumoral injection.

14. The method according to claim 13, wherein the composition comprises SEQ ID Nos. 9-10 and SEQ ID No. 13.

15. The method according to claim 13, wherein the composition comprises SEQ ID Nos. 9-10 and SEQ ID No. 14.

16. The method according to claim 14, wherein the pharmaceutical composition comprises SEQ ID Nos. 7-8, and wherein the pharmaceutical composition is administered intratumorally.

17. The method according to claim 13, wherein expression of a targeted gene is reduced by an amount selected from the group consisting of more than 20 percent, more than 30 percent, more than 40 percent, more than 50 percent, more than 60 percent, more than 70 percent, more than 80 percent, more than 90 percent, and up to 100 percent.

18. The method according to claim 13, wherein tumor volume is reduced or cancer cell levels are reduced by an amount selected from the group consisting of more than 20 percent, more than 30 percent, more than 40 percent, more than 50 percent, more than 60 percent, more than 70 percent, more than 80 percent, more than 90 percent, and up to 100 percent.

19. The method according to claim 7, wherein the subject is a mammal.

20. The method according to claim 19, wherein the subject is a human.
